(19) United States
(12) Patent Application Publication
(10) Pub. No.: US 2025/0034595 A1
(43) Pub. Date: Jan. 30, 2025

(54) TRACKABLE NUCLEIC ACID-GUIDED EDITING

(71) Applicant: Inscripta, Inc., Pleasanton, CA (US)

(72) Inventors: Brian Chaikind, Pleasanton, CA (US); Alex Hutagalung, Pleasanton, CA (US); Janine Mok, Pleasanton, CA (US)

(21) Appl. No.: 18/714,685

(22) PCT Filed: Dec. 1, 2022
(86) PCT No.: PCT/US2022/080756

§ 371 Date: May 30, 2024

Related U.S. Application Data
(60) Provisional application No. 63/285,393, filed on Dec. 2, 2021.

(51) Int. Cl. C12N 15/90; C12N 9/12; C12N 9/22; C12N 15/11

(52) U.S. Cl. CPC C12N 15/907; C12N 9/1276; C12N 9/22; C12N 15/11; C12Y 207/07049; C07K 2319/00; C12N 2310/20

(57) ABSTRACT

The present disclosure provides compositions of matter, methods and instruments for nucleic acid-guided nickase/reverse transcriptase fusion enzyme editing of nucleic acids in live mammalian cells, and for tracking of editing events.

### CROSS REFERENCE TO RELATED APPLICATIONS

This application claims the benefit of U.S. Provisional Application No. 63/285,393, filed Dec. 2, 2021, which is incorporated by reference herein in its entirety.

### FIELD OF THE INVENTION

This invention relates to compositions of matter, methods, and instruments for tracking nucleic acid-guided editing of live cells, particularly mammalian cells.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently various nucleases have been identified that allow manipulation of gene sequence, and hence gene function The nucleases include nucleic acid-guided nucleases, which enable researchers to generate permanent edits in live cells Of course, it is not only desirable to attain the highest editing rates possible in a cell population, but also to track the genomic edits in the cells, especially when multiple rounds of editing are performed and/or combinatorial libraries of edits are prepared. However, current tracking methods are inefficient and may lead to random genomic integration of tracking sequences, and/or require successive rounds of editing for targeted integration.

There is thus a need in the art of nucleic acid-guided nuclease editing for improved methods, compositions, modules, and instruments for efficient tracking of genomic edits, particularly in mammalian cells. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects, the present disclosure provides a method for performing a trackable nucleic acid-guided nickase/reverse transcriptase fusion editing in a genome of a live cell, comprising: (a) providing the live cell, where the live cell comprises a target locus and an integration locus; (b) providing a nucleic acid-guided nuclease/reverse transcriptase fusion enzyme; (c) providing a first guide RNA (gRNA) having a region of complementarity to a first sequence of the integration locus; (d) providing a second gRNA having a region of complementarity to a second sequence of the integration locus; (e) providing an editing vector, the editing vector comprising: (i) a CRISPR-enabled trackable genome engineering (CREATE) fusion (CF) editing cassette comprising from 5′ to 3′; (A) a nucleic acid sequence encoding a CFgRNA having a region of complementarity to a sequence of the target locus, and (B) a nucleic acid sequence encoding a repair template; (ii) a 5′ homology arm flanking a 5′ end of the CF editing cassette, the 5′ homology arm having homology to a third sequence of the integration locus; and (iii) a 3′ homology arm flanking a 3′ end of the CF editing cassette, the 3′ homology arm having homology to a fourth sequence of the integration locus; (f) providing conditions to allow the nucleic acid-guided nuclease/reverse transcriptase fusion enzyme, the CFgRNA, and the repair template to bind to the target locus; (g) allowing the nucleic acid-guided nuclease/reverse transcriptase fusion enzyme, the CFgRNA, and the repair template to edit the target locus; (h) providing conditions to allow the nucleic acid-guided nuclease/reverse transcriptase fusion enzyme and first and second gRNAs to bind and nick at the integration locus; and (i) allowing the CF editing cassette to integrate into the integration locus.

In some aspects, the present disclosure provides an editing system comprising one or more vectors comprising: (i) a nucleic acid sequence encoding a nucleic acid-guided nuclease/reverse transcriptase fusion enzyme; (ii) a nucleic acid sequence encoding a first gRNA having a region of complementarity to a first sequence of an integration locus in a cell; (iii) a nucleic acid sequence encoding a second gRNA having a region of complementarity to a second sequence of the integration locus; (iv) a CF editing cassette comprising from 5′ to 3′; a nucleic acid sequence encoding a CFgRNA having a region of complementarity to a sequence of a target locus in the cell, and a nucleic acid sequence encoding a repair template; (v) a 5′ homology arm flanking a 5′ end of the CF editing cassette, the 5′ homology arm having homology to a third sequence of the integration locus; and (vi) a 3′ homology arm flanking a 3′ end of the CF editing cassette, the 3′ homology arm having homology to a fourth sequence of the integration locus.

In some aspects, the present disclosure provides a vector comprising (i) a nucleic acid sequence encoding a nucleic acid-guided nuclease/reverse transcriptase fusion enzyme; (ii) a nucleic acid sequence encoding a first gRNA having a region of complementarity to a first sequence of an integration locus in a cell; (iii) a nucleic acid sequence encoding a second gRNA having a region of complementarity to a second sequence of the integration locus; (iv) a CF editing cassette comprising from 5′ to 3′; a nucleic acid sequence encoding a CFgRNA having a region of complementarity to a sequence of a target locus in the cell, and a nucleic acid sequence encoding a repair template; (v) a 5′ homology arm flanking a 5′ end of the CF editing cassette, the 5′ homology arm having homology to a third sequence of the integration locus; and (vi) a 3′ homology arm flanking a 3′ end of the CF editing cassette, the 3′ homology arm having homology to a fourth sequence of the integration locus.

These aspects and other features and advantages of the invention are described below in more detail.

### BRIEF DESCRIPTION OF THE DRAWINGS

The foregoing and other features and advantages of the present invention will be more fully understood from the following detailed description of illustrative aspects taken in conjunction with the accompanying drawings in which:

FIG. 1A is a simplified block diagram of an example of a method for trackable editing of live cells utilizing a CF editing cassette and a nucleic acid-guided nickase/reverse transcriptase fusion (“nickase-RT fusion”) enzyme, where the CF editing cassette comprises a nucleic acid sequence encoding a CFgRNA is integrated into the cell genomes for tracking of editing events. FIG. 1B is a simplified graphic depiction of the mechanism of a nucleic acid-guided nickase/reverse transcriptase fusion enzyme edit. FIG. 1C schematically depicts an example of an editing vector layout for trackable nickase-RT fusion editing of live cells comprising a CF editing cassette comprising a nucleic acid sequence encoding a CFgRNA and a nucleic acid encoding a repair template, a selectable marker, a pair of homology arms (“5′-Hom-AAVS1” and “3′-Hom-AAVS1”) flanking the CF editing cassette and selectable marker, a nucleic acid sequence encoding a nickase-RT fusion enzyme (“CFE”), and a pair of gRNAs (“gRNA1” and “gRNA2”). FIG. 1D is a simplified graphic depiction of the CF editing cassette in FIG. 1C facilitating editing of a target locus during nickase-RT fusion editing, in addition to the CF editing cassette being integrated into the integration locus (e.g., a safe harbor locus) (“AAVSI locus”) of the cell genome for tracking of the corresponding editing event. FIG. 1E schematically depicts another example of an editing vector for trackable nickase-RT fusion editing of live cells. FIG. IF depicts the frequency (y-axis) of edited cells expressing blue fluorescent protein (“BFP+”). In FIG. 1F induced pluripotent stem cells (iPSC) expressing GFP are transfected with the editing vector of FIG. 1D where the “CFgRNA+Repair Template” targets a GFP-to-BFP edit, the selectable marker is a puromycin resistance gene, and gRNA1, gRNA2, and the homology arms target various integration loci (x-axis). The BFP+ frequency IS assessed before and after selection and enrichment of the edited cells via exposure to puromycin.

FIGS. 2A-2C depict three different views of an automated multi-module cell processing instrument for performing trackable nucleic acid-guided nuclease editing employing a split protein reporter system.

FIG. 3A depicts one aspect of a rotating growth vial for use with the cell growth module described herein and in relation to FIGS. 3B-3D. FIG. 3B illustrates a perspective view of one aspect of a rotating growth vial in a cell growth module housing. FIG. 3C depicts a cut-away view of the cell growth module from FIG. 3B. FIG. 3D illustrates the cell growth module of FIG. 3B coupled to LED, detector, and temperature regulating components.

FIG. 4A depicts retentate (top) and permeate (bottom) members for use in a tangential flow filtration module (e.g., cell growth and/or concentration module), as well as the retentate and permeate members assembled into a tangential flow assembly (bottom). FIG. 4B depicts two side perspective views of a reservoir assembly of a tangential flow filtration module. FIGS. 4C-4E depict an example of a top, with fluidic and pneumatic ports and gasket suitable for the reservoir assemblies shown in FIG. 4B.

FIG. 5A depicts an example of a combination reagent cartridge and electroporation device (e.g., transformation module) that may be used in a multi-module cell processing instrument. FIG. 5B is a top perspective view of one aspect of an example of a flow-through electroporation device that may be part of a reagent cartridge. FIG. 5C depicts a bottom perspective view of one aspect of an example of a flow-through electroporation device that may be part of a reagent cartridge. FIGS. 5D-5F depict a top perspective view, a top view of a cross section, and a side perspective view of a cross section of an FTEP device useful in a multi-module automated cell processing instrument such as that shown in FIGS. 2A-2C.

FIG. 6A depicts a simplified graphic of a workflow for singulating, editing and normalizing cells in a solid wall device. FIGS. 6B-6D depict an aspect of a solid wall isolation incubation and normalization (SWIIN) module. FIG. 6E depicts the aspect of the SWIIN module in FIGS. 6B-6D further comprising a heater and a heated cover.

FIG. 7 is a simplified process diagram of an aspect of an example of an automated multi-module cell processing instrument comprising a solid wall singulation/growth/editing/normalization module for recursive and trackable cell editing—including mammalian cell editing—in a system using a nickase-RT fusion enzyme and a genome-integrating CF editing cassette.

It should be understood that the drawings are not necessarily to scale, and that like reference numbers refer to like features.

### DETAILED DESCRIPTION

All the functionalities described in connection with one aspect are intended to be applicable to the additional aspects described herein except where expressly stated or where the feature or function is incompatible with the additional aspects. For example, where a given feature or function is expressly described in connection with one aspect but not expressly mentioned in connection with an alternative aspect, it should be understood that the feature or function may be deployed, utilized, or implemented in connection with the alternative aspect unless the feature or function is incompatible with the alternative aspect.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger. Principles of Biochemistry 3^{rd} Ed., W. H. Freeman Pub., New York, N.Y.; Berg et al. (2002) Biochemistry. 5^{th} Ed., W. H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols. Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit aspects of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit aspects of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference herein in their entireties.

When a range of numbers is provided herein, the range is understood to inclusive of the edges of the range as well as any number between the defined edges of the range. For example, “between 1 and 10” includes any number between 1 and 10, as well as the number 1 and the number 10.

The term “about” means plus or minus 10% of the numerical value of the number with which it is being used. For example, “about 100” refers to numbers between (and including) 90 and 110.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. The terms “percent complementarity” or “percent complementary” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity.” can be calculated by (i) optimally base-pairing or hybridizing the two nucleotide sequences in a linear and fully extended arrangement (e.g., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%. In general, a nucleic acid includes a nucleotide sequence described as having a “percent complementarity.” or being a “percent complementary” to a specified second nucleotide sequence. For example, a nucleotide sequence may have 70%, 80%, 90%, 95%, 99%, or 100% complementarity to a specified second nucleotide sequence, indicating that, for example, 7 of 10, 8 of 10, 9 of 10, 19 of 20, 99 of 100, or 10 of 10 nucleotides, respectively, of a sequence are complementary to the specified second nucleotide sequence. For example, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

T The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

A “regulatory sequence” or “regulatory region” refers to the region of a gene where RNA polymerase and other accessory transcription modulator proteins (e.g., transcription factors) bind and interact to control transcription of the gene. Non-limiting examples of regulatory sequences or regions include promoters, enhancers, and terminators. Regulatory sequences or regions are capable of increasing or decreasing gene expression. As a result, these elements can control net protein expression from the gene.

The terms “CREATE fusion editing cassette” or “CF editing cassette” in the context of the current methods and compositions refers to a nucleic acid molecule comprising a coding sequence for transcription of a CREATE fusion gRNA, or “CFgRNA,” covalently linked to a coding sequence for transcription of a repair template for use with nickase-RT fusion enzymes. The CFgRNA and repair template are designed to bind to and facilitate editing in a nucleic acid-guided nickase/reverse transcriptase fusion system of one or both DNA strands in a target locus. In certain aspects, “CF editing cassette” refers to a nucleic acid molecule comprising a coding sequence for transcription of two gRNAs and/or two repair templates to effect editing in a nucleic acid-guided nickase/reverse transcriptase fusion system where the two gRNAs are designed to bind to and edit opposite DNA strands in a target locus. For additional information regarding traditional editing cassettes, e.g., comprising a gRNA and a repair template for use in nucleic acid-guided nuclease systems, see U.S. Pat. Nos. 9,982,278; 10,266,849; 10,240,167; 10,351,877; 10,364,442; 10,435,715; 10,465,207; 10,669,559; 10,771,284; 10,731,498; and 11,078,498, all of which are incorporated by reference herein.

The terms “CREATE fusion editing system” or “CF editing system” refer to the combination of a nucleic acid-guided nickase enzyme/reverse transcriptase fusion protein (“nickase-RT fusion”) and a CREATE fusion editing cassette (“CF editing cassette”) to effect editing in live cells.

The terms “CREATE fusion gRNA” or “CFgRNA” refer to a gRNA engineered to function with a nucleic acid-guided nickase/reverse transcriptase fusion enzyme (a “nickase-RT fusion”) where the CFgRNA is designed to bind to and facilitate editing of one or both DNA strands in a target locus of a cell genome. In certain aspects, “CREATE fusion gRNA” or “CFgRNA” refer to one of two gRNAs engineered to function with a nucleic acid-guided nickase/reverse transcriptase fusion enzyme (a “nickase-RT fusion”) where the two CFgRNAs are designed to bind to and facilitate editing of opposite DNA strands in a target locus. The two CFgRNAs specific to a target locus have regions of complementarity to one another at least at the site of the intended edit and preferably at regions 5′ and 3′ to the site of the edit. The term “complementary CFgRNAs” refers to two CFgRNAs engineered to bind to opposite DNA strands in a target locus to facilitate creation of complementary edits at a site in the target locus.

The terms “guide nucleic acid” or “guide RNA” or “gRNA” refer to a polynucleotide comprising 1) a spacer or guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on a donor DNA with a certain degree of homology with a target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity.” for the query sequence is equal to the number of identical positions between the two sequences divided by the total number of positions in the query sequence over its length (or a comparison window), which is then multiplied by 100%. When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool R (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin MA et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The term “nucleic acid” or “polynucleotide” refers to deoxyribonucleic acids (DNA) or ribonucleic acids (RNA) and polymers thereof in either single-or double-stranded form. Unless otherwise indicated, the terms encompass nucleic acids containing known analogues or natural nucleotides that have similar binding properties as the reference nucleic acid and are metabolized in a manner similar to naturally occurring nucleotides. Unless otherwise indicated, in addition to the sequence specifically stated, a particular nucleic acid sequence also implicitly encompasses conservatively modified variants thereof (e.g., degenerate codon substitutions), alleles, orthologues, SNPs, and complementary sequences. The term nucleic acid is used interchangeably with DNA, RNA, cDNA, gene, and mRNA encoded by a gene.

As used herein, “nucleic acid-guided nickase/reverse transcriptase fusion” or “nickase-RT fusion” refers to a nucleic acid-guided nickase—or nucleic acid-guided nuclease or CRISPR nuclease that has been engineered to act as a nickase rather than a nuclease that initiates double-stranded DNA breaks—where the nucleic acid-guided nickase is fused to a reverse transcriptase, which is an enzyme used to generate cDNA from an RNA template. In certain aspects, “nucleic acid-guided nickase/reverse transcriptase fusion” or “nickase-RT fusion” refers to two or more nucleic acid-guided nickases—or nucleic acid-guided nucleases or CRISPR nucleases that have been engineered to act as nickases rather than nucleases that initiate double-stranded DNA breaks—where the nucleic acid-guided nickases are fused to a reverse transcriptase.

For information regarding nickase-RT fusions see, e.g., U.S. Pat. Nos. 10,689,669 and 16/740,421.

“Nucleic acid-guided editing components” refers to one or both of a nickase-RT fusion and CREATE fusion editing cassettes (CF editing cassettes) or guide nucleic acids (CFgRNAs).

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (e.g. chromosome) and may still have interactions resulting in altered regulation.

A “PAM mutation” refers to one or more edits to a target sequence that removes, mutates, or otherwise renders inactive a protospacer adjacent motif (PAM) or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. In some aspects, a promoter is an endogenous promoter, synthetically produced, varied, or derived from a known or naturally occurring promoter sequence or other promoter sequence. In some aspects, a promoter is a constitutive promoter. In some aspects, a promoter is an inducible promoter. In some aspects, a promoter is a heterologous promoter.

A “terminator” or “terminator sequence” refers to a DNA regulatory region of a gene that signals termination of transcription of the gene to an RNA polymerase.

Without being limiting, terminators cause transcription of an operably linked nucleic acid molecule to stop.

A “coding sequence” or “coding region” refers to the region of a gene's DNA or RNA which codes for a gene product (e.g., a protein). In DNA, the coding region of a gene is flanked by the promoter sequence on the 5′ end of the template strand and the termination sequence on the 3′ end. After transcription, the coding region in an mRNA is flanked by the 5′ untranslated region (5′-UTR) and 3′ untranslated region (3′-UTR), the 5′ cap, and poly-A tail.

A “non-coding sequence” or “non-coding region” refers to the region of a gene's DNA which does not code for a protein. However, some non-coding DNA is transcribed into functional non-coding RNA molecules (e.g., transfer RNA, microRNA, siRNA, piRNA, ribosomal RNA, and regulatory RNAs). Other functional non-coding DNA include, for example, regulatory sequences of a gene that control its expression.

As used herein “gene product” refers to a biochemical material, either RNA or protein, resulting from expression of a gene. In some aspects, a gene product is an RNA molecule, e.g., transfer RNA, microRNA, siRNA, piRNA, ribosomal RNA, or regulatory RNA. In some aspects, the gene product is a protein. In some aspects, the gene product is an enzyme. In some aspects, the gene product is a membrane protein. In some aspects, the gene product is a protein involved in the expression of a gene. In some aspects, the gene product is a transcription factor. In some aspects, the gene product is a coactivator protein. In some aspects, the gene product is a corepressor protein. In some aspects, the gene product is a chromatin-binding protein.

As used herein, the terms “protein,” “peptide,” and “polypeptide” are used interchangeably herein and refer to a polymer of amino acid residues. In some aspects, proteins are made up entirely of amino acids transcribed by any class of any RNA polymerase I, II or III.

As used herein, the term “repair template” in the context of a CREATE fusion editing system employing a nickase-RT fusion enzyme refers to a nucleic acid (.e.g., a ribonucleic acid) that is designed to serve as a template (including a desired edit) to be incorporated into target DNA via reverse transcription (e.g., by reverse transcriptase).

As used herein, the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other aspects, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS: confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). In some aspects, a selectable marker comprises an antibiotic resistance gene. In some aspects, a selectable marker comprises a puromycin resistance gene. “Selective medium” as used herein refers to cell growth medium to which has been added a chemical compound or biological moiety that selects for or against selectable markers.

A “locus” refers to a fixed position in a genome. In some aspects, a locus comprises a coding region. In some aspects, a locus comprises a non-coding region. In some aspects, a locus comprises a gene. In an aspect, a locus comprises at least 1nucleotide. In an aspect, a locus comprises at least 10 nucleotides. In an aspect, a locus comprises at least 25 nucleotides. In an aspect, a locus comprises at least 50 nucleotides. In an aspect, a locus comprises at least 100 nucleotides. In an aspect, a locus comprises at least 250 nucleotides. In an aspect, a locus comprises at least 500 nucleotides. In an aspect, a locus comprises at least 1000 nucleotides. In an aspect, a locus comprises at least 2500 nucleotides. In an aspect, a locus comprises at least 5000 nucleotides.

The terms “target genomic DNA locus”, “target locus”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus. In some aspects, a target locus refers to a position in a genome targeted to be edited by the nucleic acid-guided nuclease/reverse transcriptase fusion enzyme and the CF editing cassette. In some aspects, a target locus comprises a gene, including its regulatory regions and coding regions. In some aspects, a target locus comprises a regulatory region of a gene, e.g., a promoter region or a terminator region.

In some aspects, an “integration locus” refers to a position in a genome targeted for the integration of a CF editing cassette. In some aspects, an integration locus comprises a coding region. In some aspects, an integration locus comprises a non-coding region. In some aspects, an integration locus comprises a “safe harbor locus.” A “safe harbor locus” as used herein refers to an intergenic region that has a reduced potential for the CF editing cassette integration adversely affecting genes neighboring the integrated CF editing cassette.

The term “gene” refers to a nucleic acid region which includes a coding region operably linked to a suitable regulatory region capable of regulating the expression of a gene product (e.g., a polypeptide or functional RNA) in some manner. Genes include untranslated regulatory regions (e.g., promoters, enhancers, repressors, etc.) in the DNA before (upstream) and after (downstream) the coding region (open reading frame, ORF), and, where applicable, intervening sequences (e.g., introns) between individual coding regions (e.g., exons).

The term “variant” may refer to a polypeptide or polynucleotide that differs from a reference polypeptide or polynucleotide. A typical variant of a polypeptide differs in amino acid sequence from another reference polypeptide. Generally, differences may be limited so that the sequences of the reference polypeptide and the variant are closely similar overall (e.g., at least 90% identical) and, in many regions, identical. A variant and reference polypeptide may differ in amino acid sequence by one or more modifications (e.g., substitutions, additions, and/or deletions). A variant of a polypeptide may be a conservatively modified variant (e.g., at least 90% identical to the reference polypeptide). A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In the present disclosure, a single vector may include a coding sequence for a nickase-RT fusion enzyme and a CF editing cassette and/or CFgRNA sequence to be transcribed. In other aspects, however, two vectors—e.g., an engine vector comprising the coding sequence for the nickase-RT fusion enzyme, and an editing vector, comprising the CFgRNA sequence to be transcribed—may be used.

As used herein, a “mutation” refers to an inheritable genetic modification introduced into a gene to alter the expression or activity of a product encoded by the gene. In some aspects, “mutation,” “modification,” and “edit” may be used interchangeably in the present disclosure. In some aspects, a modification can be in any sequence region of a gene, for example, in a promoter, 5′ UTR, exon, 3′ UTR, or terminator region. In some aspects, a modification can be in the regulatory region of a gene. In some aspects, a modification can be in the coding region of a gene. In some aspects, a modification reduces, inhibits, or eliminates the expression or activity of a gene product. In some aspects, a modification increases, elevates, strengthens, or augments the expression or activity of a gene product.

In some aspects, a mutation, or modification is a “non-natural” or “non-naturally occurring” mutation or modification. As used herein, a “non-natural” or “non-naturally occurring” mutation or modification refers to a non-spontaneous mutation or modification generated via human intervention, and does not correspond to a spontaneous mutation or modification generated without human intervention. Non-limiting examples of human intervention include mutagenesis (e.g., chemical mutagenesis, ionizing radiation mutagenesis) and targeted genetic modifications (e.g., nucleic-acid guided nuclease-based methods, CREATE fusion-based methods, CRISPR-based methods, TALEN-based methods, zinc finger-based methods). Non-natural mutations or modifications and non-naturally occurring mutations or modifications do not include spontaneous mutations that arise naturally (e.g., via aberrant DNA replication).

Several types of mutations or modifications are known in the art. In some aspects, a mutation or modification comprises an insertion. An “insertion” refers to the addition of one or more nucleotides or amino acids to a given polynucleotide or amino acid sequence, respectively, as compared to an endogenous reference polynucleotide or amino acid sequence.

In some aspects, a mutation or modification comprises a deletion. A “deletion” refers to the removal of one or more nucleotides or amino acids to a given polynucleotide or amino acid sequence, respectively, as compared to an endogenous reference polynucleotide or amino acid sequence.

In some aspects, a mutation or modification comprises a substitution or a swap. A “substitution” or “swap” refers to the replacement of one or more nucleotides or amino acids to a given polynucleotide or amino acid sequence, respectively, as compared to an endogenous reference polynucleotide or amino acid sequence. In some aspects, a “substitution allele” refers to a nucleic acid sequence at a particular locus comprising a substitution.

In some aspects, a mutation or modification comprises an inversion. An “inversion” refers to when a segment of a polynucleotide or amino acid sequence is reversed end-to-end. In some aspects, a mutation or modification provided herein comprises a mutation selected from the group consisting of an insertion, a deletion, a substitution, and an inversion. In some aspects, a mutation or modification provided herein comprises an insertion. In some aspects, a mutation or modification provided herein comprises a deletion. In some aspects, a mutation or modification provided herein comprises a substitution. In some aspects, a mutation or modification provided herein comprises an inversion.

In some aspects, a mutation or modification comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations.

Mutations or modifications in coding regions of genes (e.g., exonic mutations) can result in a truncated protein or polypeptide when a mutated messenger RNA (mRNA) is translated into a protein or polypeptide. In some aspects, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In some aspects, this disclosure provides a mutation that results in a premature stop codon in an mRNA transcript of an endogenous gene.

As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.” In some aspects, multiple (e.g., 2, 3, 4, 5, 6, 7, 8, 9, 10, or more than 10) premature stop codons are introduced.

In some aspects, a mutation or modification provided herein comprises a null mutation. As used herein, a “null mutation” refers to a mutation that confers a decreased function or complete loss-of-function for a protein encoded by a gene comprising the mutation, or, alternatively, a mutation that confers a decreased function or complete loss-of-function for a small RNA encoded by a genomic locus. A null mutation can cause lack or decrease of mRNA transcript production, small RNA transcript production, protein function, or a combination thereof. As used herein, a “null allele” refers to a nucleic acid sequence at a particular locus where a null mutation has conferred a decreased function or complete loss-of-function to the allele.

In some aspects, a “synonymous edit” or “synonymous substitution” is the substitution of one base for another in an exon of a gene coding for a protein, such that the produced amino acid sequence is not modified. This is possible because the genetic code is “degenerate”, meaning that some amino acids are coded for by more than one three-base-pair codon; since some of the codons for a given amino acid differ by just one base pair from others coding for the same amino acid, a mutation that replaces the “normal” base by one of the alternatives will result in incorporation of the same amino acid into the growing polypeptide chain when the gene is translated.

In some aspects, “codon optimization” refers to experimental approaches designed to improve the codon composition of a recombinant gene based on various criteria without altering the amino acid sequence. This is possible because most amino acids are encoded by more than one codon. Codon optimization may be used to improve gene expression and increase the translation efficiency of a gene of interest by accommodating for codon bias of the host organism. In some aspects, a nucleic acid molecule provided herein encodes a polypeptide that is codon optimized for a prokaryote. In some aspects, a nucleic acid molecule provided herein encodes a polypeptide that is codon optimized for a eukaryote. In some aspects, a nucleic acid molecule provided herein encodes a polypeptide that is codon optimized for a mammalian cell. In some aspects, a nucleic acid molecule provided herein encodes a polypeptide that is codon optimized for an archaeal cell.

The present disclosure includes methods of trackable nucleic acid-guided nuclease editing in cell populations, e.g., prokaryotic, archaeal, and eukaryotic cells. In some aspects, the cells include mammalian cells. In some aspects, the cells include bacterial or fungal cells.

In some aspects, a mutation or modification provided herein can be positioned in any part of a gene. In some aspects, a mutation or modification provided herein can be positioned in the coding region of a gene. In some aspects, a mutation or modification provided herein can be positioned in the non-coding region of a gene. In some aspects, a mutation or modification provided herein can be positioned in the regulatory region of a gene. In some aspects, a mutation or modification provided herein is positioned within an exon of a gene. In some aspects, a mutation or modification provided herein is positioned within an intron of a gene. In a further aspect, a mutation or modification provided herein is positioned within a 5′-untranslated region (UTR) of a gene. In still another aspect, a mutation or modification provided herein is positioned within a 3′-UTR of a gene. In yet another aspect, a mutation or modification provided herein is positioned within a promoter of a gene. In yet another aspect, a mutation or modification provided herein is positioned within a terminator of a gene.

The present disclosure relates to methods and compositions for improved tracking of nucleic acid-guided nuclease editing. With the present compositions and methods, targeted editing and tracking of the intended edit(s) is facilitated using (i) a single fusion protein and (ii) a corresponding CF editing cassette (“CREATE fusion editing cassette,” defined infra) comprising a nucleic acid sequence encoding a CFgRNA (“CREATE fusion guide RNA”) and a nucleic acid sequence encoding a repair template, flanked by homology arms for incorporation of the CF editing cassette at an integration locus of a cellular genome, and (iii) guide RNA(s) (gRNA(s)) targeting the integration locus. The homology arms and gRNAs are designed to integrate the CF editing cassette at the integration locus. The CF editing cassette is designed to edit one or both DNA strands at a target locus of the cellular genome. The fusion protein—e.g., a nickase/reverse transcriptase (“nickase-RT fusion”)—retains certain characteristics of nucleic acid-directed nucleases (e.g., the binding specificity and ability to nick or cleave one or more DNA strands in a targeted manner) combined with another enzymatic activity, namely, reverse transcriptase activity. When introduced into cells along with a corresponding CF editing cassette, the same nickase-RT fusion that enables editing further facilitates integration of the CF editing cassette (including the CFgRNA and repair template) at an integration locus for tracking. Accordingly, a single enzyme enables both editing and tracking of the intended edit(s), while integration of the CF editing cassette further eliminates the need for additional barcode sequences, thereby simplifying the editing process.

In certain aspects, the nickase-RT fusion is introduced into the cells using a DNA molecule coding for the nickase-RT fusion separately or linked to the CF editing cassette, or the nickase-RT fusion may be introduced separately in protein form or as part of a complex. In addition to the nickase-RT fusion, the CF editing cassette comprising the nucleic acid sequence encoding the CFgRNA and the nucleic acid sequence encoding the repair template is utilized. The reverse transcriptase portion of the nickase-RT fusion uses the CF editing cassette to synthesize and reverse transcribe a “flap” at a target locus specified by the nickase portion of the nickase-RT fusion, and the edited flap may be resolved into the genome via endogenous repair mechanisms, e.g., homology-directed repair (HDR), by recombination pathways, or other DNA repair pathways.

In certain aspects, the CF editing cassette is introduced into the cells using a DNA molecule comprising the CF editing cassette and a pair of homology arms flanking the CF editing cassette, where each of the homology arms has complementarity to a sequence/region of an integration locus of the cell genome. In addition to the homology arms, a pair of gRNAs recognized by the nickase-RT fusion and having complementary to the integration locus is utilized. The nickase portion of the nickase-RT fusion uses the pair of gRNAs to introduce staggered single-stranded cuts, or “nicks,” in the integration locus, which may be repaired via HDR mechanisms utilizing the CF editing cassette as a repair template, thus integrating the CF editing cassette into the integration locus.

Thus, certain aspects of the present disclosure provide a method for performing nucleic acid-guided nickase/reverse transcriptase fusion editing in a genome of a live cell, comprising: (a) providing the live cell, where the live cell comprises a target locus and an integration locus; (b) providing a nucleic acid-guided nuclease/reverse transcriptase fusion enzyme; (c) providing a first gRNA having a region of complementarity to a first sequence within the integration locus; (d) providing a second gRNA having a region of complementarity to a second sequence within the integration locus; (e) providing an editing vector, the editing vector comprising (i) a CF editing cassette comprising from 5′ to 3′; (A) a nucleic acid sequence encoding a CFgRNA having a region of complementarity to a sequence of the target locus, where the CFgRNA comprises a spacer region (e.g., a guide sequence) and a structural region, the structural region recognized by a corresponding nuclease or nickase (e.g., a scaffold); and (B) a nucleic acid sequence encoding a repair template comprising from 5′ to 3′ an optional post-edit homology region, an edit, an optional nick-to-edit region, and a primer binding site (PBS) capable of binding to a nicked target DNA; (ii) a 5′ homology arm flanking a 5′ end of the CF editing cassette, the 5′ homology arm having homology to a third sequence of the integration locus; and (iii) a 3′ homology arm flanking a 3′ end of the CF editing cassette, the 3′ homology arm having homology to a fourth sequence of the integration locus; (f) providing conditions to allow the nucleic acid-guided nuclease/reverse transcriptase fusion enzyme, the CFgRNA, and the repair template to bind to the target locus; (g) allowing the nucleic acid-guided nuclease/reverse transcriptase fusion enzyme, the CFgRNA and the repair template to edit the target locus; (h) providing conditions to allow the nucleic acid-guided nuclease/reverse transcriptase fusion enzyme and the first and second gRNAs to bind to the integration locus; (i) and allowing the CF editing cassette to integrate into the integration locus.

In some aspects, the CF editing cassette further comprises a nucleic acid sequence encoding an RNA stabilization moiety that is linked to the 3′ end of the repair template via a linker region to stabilize the cassette and improve target nicking or cleavage efficiency without inducing off-target activity. In some aspects, the RNA stabilization moiety is an RNA G-quadraplex region, an RNA hairpin, an RNA pseudoknot, or an exoribonuclease resistant RNA.

In some aspects, the integrated CF editing cassette in the integration locus facilitates tracking of the edit to the target locus.

In some aspects, the integration of the CF editing cassette is tracked or analyzed via RNA sequencing (e.g., transcriptome sequencing) or genomic sequencing.

In some aspects, the nucleic acid-guided nuclease/reverse transcriptase fusion enzyme comprises, in order from amino terminus to carboxy terminus, a nucleic acid-guided nickase and a reverse transcriptase. In some aspects, the nucleic acid-guided nuclease/reverse transcriptase fusion enzyme comprises, in order from amino terminus to carboxy terminus, a reverse transcriptase and a nucleic acid-guided nickase. In some aspects, a nucleic acid-guided nuclease/reverse transcriptase fusion enzyme comprises a linker between the nucleic acid-guided nuclease and the reverse transcriptase. In some aspects, the linker comprises at least 1, at least 2, at least 3, at least 4, at least 5, at least 6, at least 7, at least 8, at least 9, or at least 10 amino acid residues.

In some aspects, a nucleic acid sequence encoding the nucleic acid-guided nuclease/reverse transcriptase fusion enzyme is introduced into the cell on the same editing vector as the CF editing cassette and/or the homology arms. In some aspects, a nucleic acid sequencing encoding the nucleic acid-guided nuclease/reverse transcriptase fusion enzyme is introduced into the cell on a different vector as the CF editing cassette. In some aspects, the nucleic acid-guided nuclease/reverse transcriptase fusion enzyme is introduced into the cell as a protein or complex (e.g., a ribonucleoprotein complex). In some aspects, a nucleic acid sequence encoding the nucleic acid-guided nuclease/reverse transcriptase fusion enzyme is inserted into a vector backbone, such as a pUC19 vector backbone, prior to introduction into the cell. In some aspects, a nucleic acid sequence encoding the nucleic acid-guided nuclease/reverse transcriptase fusion enzyme is introduced into the cell on a linear or circular plasmid. In some aspects, a nucleic acid sequence encoding the nucleic acid-guided nuclease/reverse transcriptase fusion enzyme is under the control of a constitutive or inducible promoter at a 5′ end thereof.

In various aspects of the various methods described herein, fusion proteins are sometimes described in certain amino to carboxy terminus sequences of their protein components. Various aspects of the methods disclosed herein employ fusion proteins that comprise the same protein components ordered in a different sequence.

In some aspects of the method, the nuclease portion of the nickase/reverse transcriptase fusion enzyme includes a MAD-series nickase or a variant (e.g., orthologue) thereof. In some aspects, the nickase includes a MADI, MAD2, MAD3,

MAD4, MAD5, MAD6, MAD7R, MAD8, MAD9, MADI0, MADII, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nickase, variants thereof, and/or combinations thereof. See, for example, U.S. Patent Application Publication No. 2020/0231987).

In some aspects of the method, the nuclease portion of the nickase/reverse transcriptase fusion enzyme includes a Cas9 nickase or a variant thereof. In some aspects of the method, the nuclease portion of the nickase/reverse transcriptase fusion enzyme includes a Cpfl nickase or a variant thereof.

In some aspects of the method, the reverse transcriptase portion of the nickase/reverse transcriptase fusion enzyme is selected from an HIV-1 reverse transcriptase, an M-MLV reverse transcriptase, an AMV reverse transcriptase, a Tf1 reverse transcriptase, and an RSV reverse transcriptase.

In some aspects, a nucleic acid sequence encoding the first gRNA and/or a nucleic acid sequence encoding the second gRNA are introduced into the cell on the same editing vector as the CF editing cassette (encoding the CFgRNA) and/or the homology arms. In some aspects, a nucleic acid sequence encoding the first gRNA and/or a nucleic acid sequence encoding the second gRNA are introduced into the cell on the same vector as a nucleic acid sequence encoding the nucleic acid-guided nuclease/reverse transcriptase fusion enzyme. In some aspects, a nucleic acid sequence encoding the first gRNA and/or a nucleic acid sequence encoding the second gRNA are under the control of a constitutive or inducible promoter at 5′ ends thereof. In some aspects, the nick-to-nick distance generated by the first gRNA and

the second gRNA in an integration locus is between 10 nucleotides and 5,000 nucleotides in length, between 10) nucleotides and 2,500 nucleotides in length, between 10) nucleotides and 2,000 nucleotides in length, between 10) nucleotides and 1,000 nucleotides in length, or between 10) and 100 nucleotides in length. In some aspects, the nick-to-nick distance is between 10) nucleotides and 4,000 nucleotides in length, between 50 nucleotides and 3,000 nucleotides in length, between 100 nucleotides and 2,500 nucleotides in length, between 200 nucleotides and 2,000 nucleotides in length, or between 500 and 1,000 nucleotides in length.

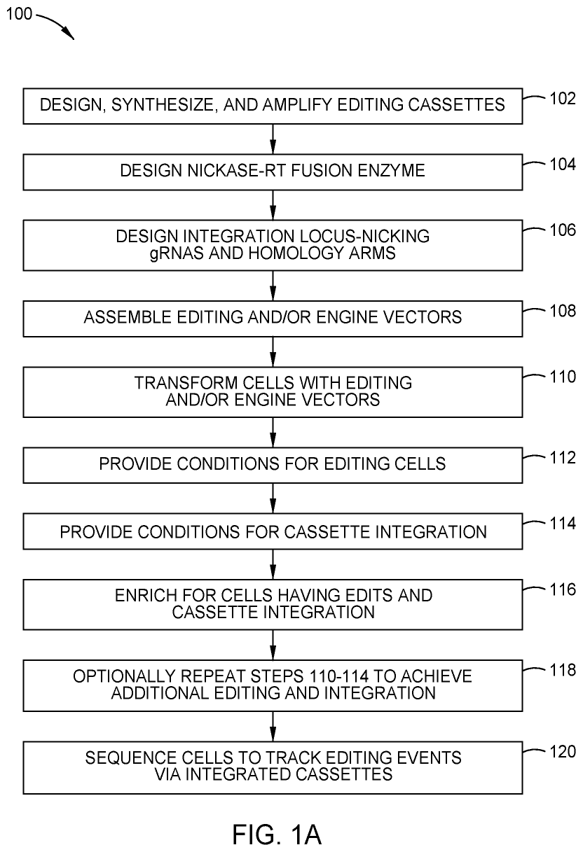

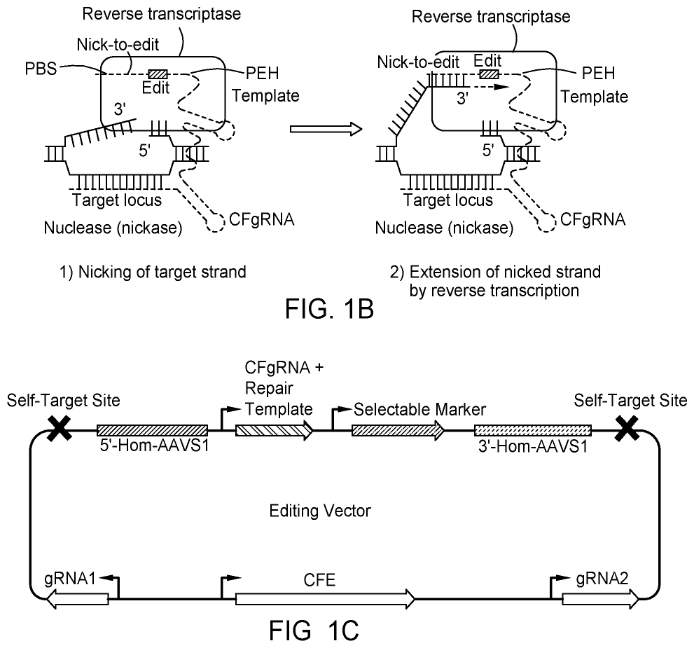

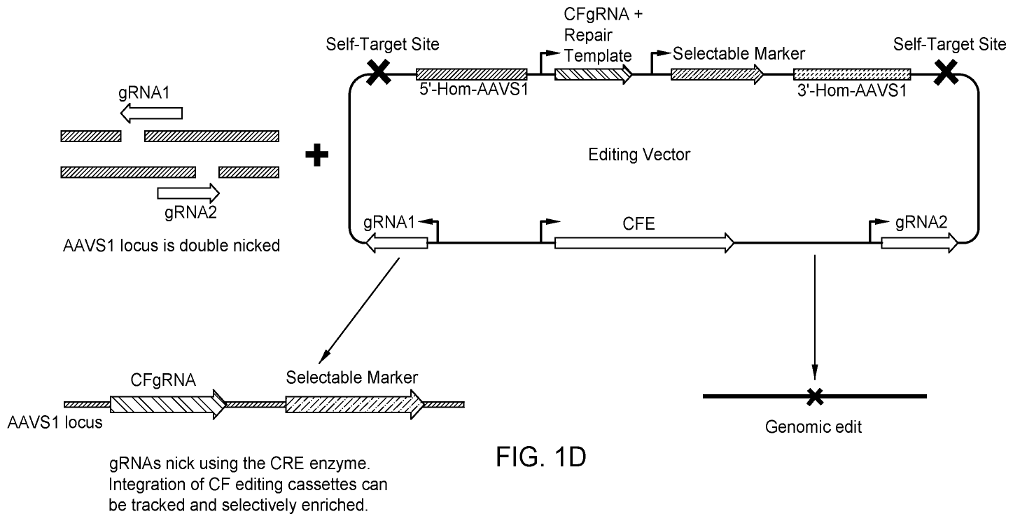

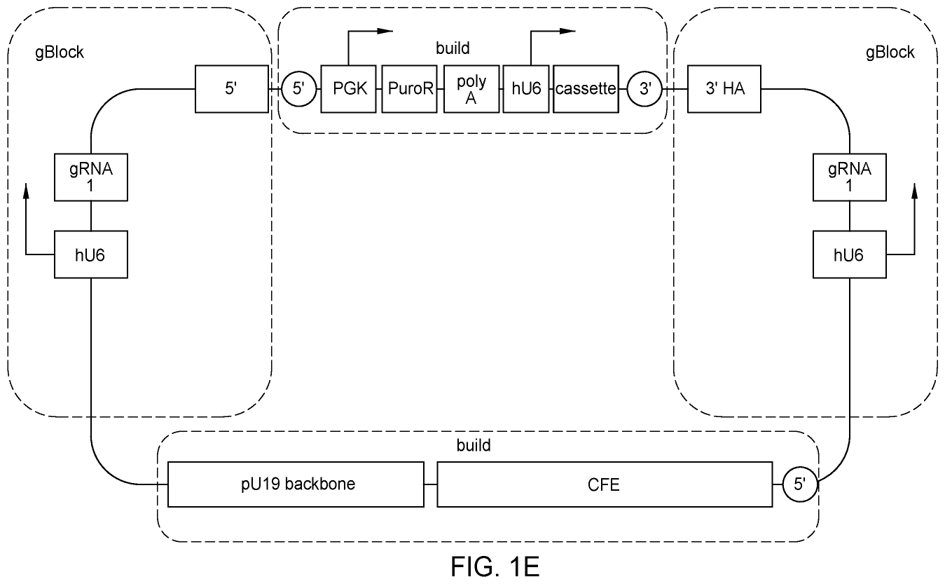

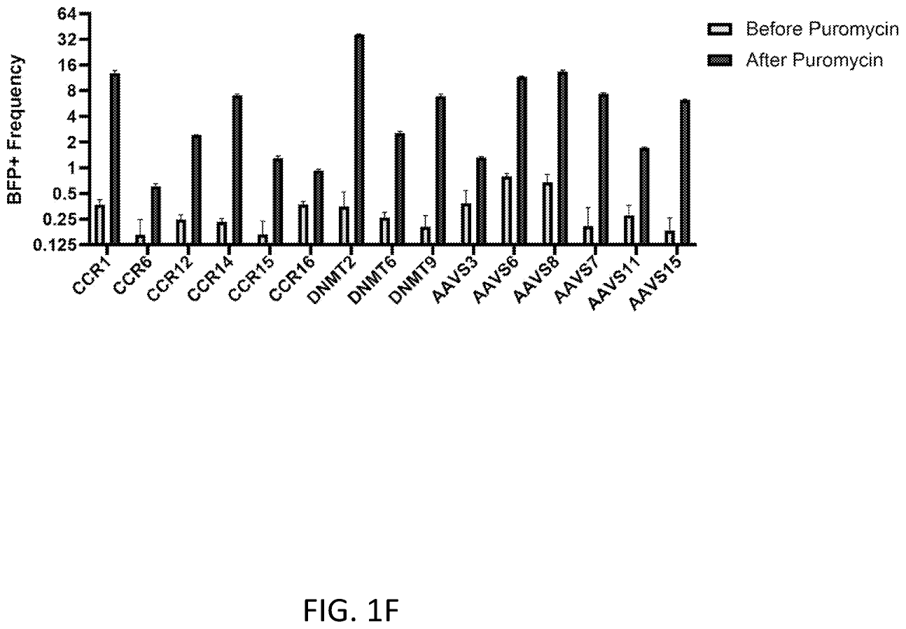

In some aspects, the editing vector comprising the CF editing cassette and homology arms is a self-cutting or self-nicking vector and further comprises self-targeting sequences having complementarity to a first and/or second gRNA. In some aspects, each of two self-targeting sequences are located at each end of a region in the editing cassette comprising the 5′ homology arm, the CF editing cassette, the selectable marker, and the 3′ homology arm, as depicted in FIG. 1D. In some aspects, each of two self-targeting sequences are located at each end of a region in the editing cassette comprising the 5′ homology arm, the CF editing cassette, and the 3′ homology arm.

In some aspects of the method, the editing vector or CF editing cassette further comprises a selectable marker located upstream or downstream of the nucleic acid sequence encoding the CFgRNA, which can be integrated into the integration locus along with the nucleic acid sequence encoding the CFgRNA. The selectable marker can be utilized for selective enrichment of edited cells. In some aspects, the selectable marker comprises an antibiotic resistance gene or encodes a fluorescent protein. In some aspects, the selectable marker comprises a puromycin resistance (PuroR) gene. In some aspects, the nucleic acid sequence encoding the CFgRNA and/or the selectable marker is under the control of a constitutive or inducible promoter at a 5′ end thereof, and a terminator sequence at a 3′ end thereof. For example, in some aspects, the CF editing cassette comprises a promoter at the 5′end of the nucleic acid sequence encoding the CFgRNA, and a terminator at the 3′ end of the nucleic acid sequence encoding the repair template.

In some aspects, the 5′ homology arm and/or the 3′ homology arm are between 10 nucleotides and 2,000 nucleotides in length, between 10 nucleotides and 1,500 nucleotides in length, between 10 nucleotides and 1,000 nucleotides in length, or between 10 nucleotides and 100 nucleotides in length. In some aspects, the 5′ homology arm and/or the 3′ homology arm are between 20 nucleotides and 2,000 nucleotides in length, between 50) nucleotides and 1,500 nucleotides in length, between 100 nucleotides and 1,000 nucleotides in length, between 200 nucleotides and 800 nucleotides in length, or between 400 nucleotides and 600 nucleotides in length.

In some aspects of the method, the integration locus facilitates stable integration of the CF editing cassette without significant impact on cell growth or function. In some aspects, an integration locus comprises a non-coding region. In some aspects, an integration locus is a safe harbor locus. In some aspects, where a plurality of CF editing cassette integrations are performed, the CF editing cassettes are embedded into one or more clustered neutral safe harbor loci. In some aspects, a safe harbor locus does not comprise a coding sequence. In some aspects, a safe harbor locus does not comprise a gene. In some aspects, a safe harbor locus is positioned on the same chromosome (in a eukaryote) as a target locus. In some aspects, a safe harbor locus is positioned on a different chromosome (in a eukaryote) as a target locus.

In some aspects, the integration locus is located within a coding region (e.g., exon). In some aspects, the integration locus is located within a noncoding region (e.g., intron or intergenic region). In some aspects, the integration locus comprises an adeno-associated virus site 1 (AAVS1), a chemokine (C-C motif) receptor 5 (CCR5) gene, a DNA methyltransferase 3B (DNMT3b) gene, or an orthologue of the Rosa26 locus. In some aspects, the integration locus comprises a chemokine (C-C motif) receptor 1(CCR1) gene, a chemokine (C-C motif) receptor 6 (CCR6) gene, a chemokine (C-C motif) receptor 12 (CCR12) gene, a chemokine (C-C motif) receptor 14 (CCR14) gene, a chemokine (C-C motif) receptor 15 (CCR15) gene, a chemokine (C-C motif) receptor 16 (CCR16) gene, a DNA methyltransferase 2 (DNMT2) gene, a DNA methyltransferase 6 (DNMT6) gene, a DNA methyltransferase 9 (DNMT9) gene, a adeno-associated virus site 3 (AAVS3), adeno-associated virus site 6 (AAVS6), an adeno-associated virus site 8 (AAVS8), an adeno-associated virus site 7 (AAVS7), an adeno-associated virus site 11 (AAVS11), or an adeno-associated virus site 15 (AAVS15).

In some aspects of the method, a region of the CF editing cassette, e.g., the region encoding the repair template, further comprises an edit (e.g., 1, 2, 3, 4, 5, or up to 10 edits) to immunize the target locus to prevent re-nicking. Because after the target locus is edited, the nucleic acid-guided polypeptide could further edit the edited target locus, methods of immunizing the target locus to prevent a subsequent edit can be performed. As discussed herein, in some aspects, an edit to immunize the target locus to prevent re-nicking is one that alters the proto-spacer adjacent motif (PAM) (or other element) such that binding at the edited target site by the nucleic acid-guided polypeptide (e.g., nuclease, nickase, inactive nuclease or inactive nickase) is impaired or prevented.

In some aspects of the method, the nick-to-edit region of the CF editing cassette, e.g., the region encoding the repair template, is between 2 nucleotides and 250 nucleotides in length, between 5 nucleotides and 150 nucleotides in length, or between 1 nucleotide and 150 nucleotides in length. In some aspects of this method, the nick-to-edit region of the CF editing cassette is up to 10,000 nucleotides in length, or up to 3,000 nucleotides in length.

In some aspects, the region of complementarity between the CF editing cassette, e.g., the region encoding the CFgRNA, and the target locus is between 4 nucleotides and 120 nucleotides in length, between 5 nucleotides and 80 nucleotides in length, between 6 nucleotides and 60 nucleotides in length, e.g., between 1 nucleotide and 10 nucleotides in length, between 10 nucleotides and 20 nucleotides in length, between 20 nucleotides and 50 nucleotides in length, or between 50 nucleotides and 100 nucleotides in length.

In some aspects, the edit region of the CF editing cassette, e.g., region encoding the repair template, is between 1 nucleotide and 750 nucleotides in length, between 1 nucleotide and 500 nucleotides in length, or between 1 nucleotide and 150 nucleotides in length, e.g., between 1 nucleotide and 10 nucleotides in length, between 10 nucleotides and 20 nucleotides in length, between 20 nucleotides and 50 nucleotides in length, between 50 nucleotides and 100 nucleotides in length, between 100 nucleotides and 250 nucleotides in length, between 250 nucleotides and 500 nucleotides in length, or between 500 nucleotides and 750 nucleotides in length.

In some aspects of the method, a post-edit homology region of the CF editing cassette, e.g., in the region encoding the repair template, is between 1 nucleotide and 50 nucleotides in length, between 2 nucleotides and 50 nucleotides in length, between 4 nucleotides and 40 nucleotides in length, or between 5 nucleotides and 25 nucleotides in length. In some aspects, the post-edit homology region of the CF editing cassette is between 1 nucleotide and 5 nucleotides in length, between 5 nucleotides and 10 nucleotides in length, between 10 nucleotides and 20 nucleotides in length, or between 20 nucleotides and 50 nucleotides in length.

In some aspects, the modification or edit created in the target locus includes one or more nucleotide swaps or substitutions in the target locus. In some aspects, the modification or edit created in the target locus includes two or more nucleotide swaps or substitutions in the target locus. In some aspects, the modification or edit created in the target locus includes three or more nucleotide swaps or substitutions in the target locus. In some aspects, the modification or edit created in the target locus includes four or more nucleotide swaps or substitutions in the target locus. In some aspects, the modification or edit created in the target locus includes five or more nucleotide swaps or substitutions in the target locus. In some aspects, the modification or edit created in the target locus includes ten or more nucleotide swaps or substitutions in the target locus.

In some aspects, the modification or edit created in the target locus is an insertion in the target locus.

In some aspects, a region of the CF editing cassette, e.g., the region encoding the repair template, is designed to provide an insertion of between 1 nucleotide and 750 nucleotides at the target site. In some aspects, the CF editing cassette is designed to provide an insertion of between 1 nucleotide and 10 nucleotides, between 10 nucleotides and 20 nucleotides, between 20 nucleotides and 50 nucleotides, between 50 nucleotides and 100 nucleotides, between 100 nucleotides and 200 nucleotides, between 200 nucleotides and 500 nucleotides or between 250 nucleotides and 750 nucleotides at the target site.

In some aspects, the modification or edit created in the target locus is an insertion of recombinase sites, protein degron tags, promoters, terminators, alternative-splice sites, CpG islands, etc.

In some aspects, the modification or edit created in the target locus is a deletion in the target locus.

In some aspects, a region of the CF editing cassette, e.g., the region encoding the repair template, is designed to provide a deletion of between 1 nucleotide and 750 nucleotides at the target site. In some aspects, the CF editing cassette is designed to provide a deletion of between 1 nucleotide and 10 nucleotides, between 10 nucleotides and 20 nucleotides, between 20 nucleotides and 50 nucleotides, between 50 nucleotides and 100 nucleotides, between 100 nucleotides and 200 nucleotides, between 200 nucleotides and 500 nucleotides or between 250 nucleotides and 750 nucleotides at the target site.

In some aspects, the modification or edit created in the target locus is a deletion of introns, exons, repetitive elements, promoters, terminators, insulators, CpG islands, non-coding elements, retrotransposons, etc.

In some aspects, the modification or edit created in the target locus comprises several types of edits and/or comprises more than one of one or more types of edits. For example, in some aspects, the edit comprises two or more nucleotide swaps or substitutions (e.g., 2, 3, 4, 5, or between 1 and 20 nucleotide swaps or substitutions), some or all of which can be adjacent to each other or nonadjacent to each other. In some aspects, the modification or edit comprises one or more nucleotide swaps or substitutions (e.g., 2, 3, 4, 5, or between 1 and 20 nucleotide swaps or substitutions) and an insertion of one or more nucleotides (e.g., 2, 3, 4, 5, or between 1 and 20 nucleotides). In some aspects, the modification or edit comprises one or more nucleotide swaps or substitutions (e.g., 2, 3, 4, 5, or between 1 and 20 nucleotide swaps or substitutions) and a deletion of one or more nucleotides or substitutions (e.g., 2, 3, 4, 5, or between 1 and 20 nucleotides).

In some aspects, the modification or edit created in the target locus is in a coding region in the target locus. In some aspects, the modification or edit created in the target locus is in a noncoding region in the target locus. In some aspects, the modification or edit created in the target locus is within a regulatory region of a gene. In some aspects, the modification or edit created in the target locus is within a promoter region of a gene. In some aspects, the modification or edit created in the target locus is within a coding region of a gene.

In some aspects, the present disclosure provides a library of vector or plasmid backbones and/or a library of CF editing cassettes to be transformed into cells. In some aspects, one or more CF editing cassettes in the library of CF editing cassettes each encodes a different CFgRNA targeting a different target locus within the cell genome, and/or a different repair templates. In some aspects, the utilization of a library of CF editing cassettes and/or a library of vector or plasmid backbones, enables combinatorial or multiplex editing in the cells.

In some aspects, the present disclosure provides a method for performing a trackable nucleic acid-guided nickase/reverse transcriptase fusion editing in a genome of a live cell, comprising: (a) providing the live cell, where the live cell comprises a target locus and an integration locus; (b) providing a nucleic acid-guided nuclease/reverse transcriptase fusion enzyme; (c) providing a first guide RNA (gRNA) having a region of complementarity to a first sequence of the integration locus; (d) providing a second gRNA having a region of complementarity to a second sequence of the integration locus; (e) providing an editing vector, the editing vector comprising: (i) a CF editing cassette comprising from 5′ to 3′: (A) a nucleic acid sequence encoding a CFgRNA having a region of complementarity to a sequence of the target locus, and (B) a nucleic acid sequence encoding a repair template; (ii) the editing vector further comprising a 5′ homology arm flanking a 5′ end of the CF editing cassette, the 5′ homology arm having homology to a third sequence of the integration locus; and (iii) a 3′ homology arm flanking a 3′ end of the CF editing cassette, the 3′ homology arm having homology to a fourth sequence of the integration locus; (f) providing conditions to allow the nucleic acid-guided nuclease/reverse transcriptase fusion enzyme, the CFgRNA, and the repair template to bind to the target locus; (g) allowing the nucleic acid-guided nuclease/reverse transcriptase fusion enzyme, the CFgRNA, and the repair template to edit the target locus; (h) providing conditions to allow the nucleic acid-guided nuclease/reverse transcriptase fusion enzyme and first and second gRNAs to bind and nick at the integration locus; and (i) allowing the CF editing cassette to integrate into the integration locus. In some aspects, the method further comprises sequencing the genome or a transcriptome of the cell to track for integration of the CF editing cassette, the integration of the CF editing cassette representing a nucleic acid-guided nickase/reverse transcriptase fusion editing event. In some aspects, the method further comprises selecting and enriching for cells having an integrated CF editing cassette.

In some aspects, the present disclosure provides an editing system comprising one or more vectors comprising: (i) a nucleic acid sequence encoding a nucleic acid-guided nuclease/reverse transcriptase fusion enzyme; (ii) a nucleic acid sequence encoding a first gRNA having a region of complementarity to a first sequence of an integration locus in a cell; (iii) a nucleic acid sequence encoding a second gRNA having a region of complementarity to a second sequence of the integration locus; (iv) a CF editing cassette comprising from 5′ to 3′: a nucleic acid sequence encoding a CFgRNA having a region of complementarity to a sequence of a target locus in the cell, and a nucleic acid sequence encoding a repair template; (v) a 5′ homology arm flanking a 5′ end of the CF editing cassette, the 5′ homology arm having homology to a third sequence of the integration locus; and (vi) a 3′ homology arm flanking a 3′ end of the CF editing cassette, the 3′ homology arm having homology to a fourth sequence of the integration locus.

In some aspects, the present disclosure provides a vector comprising (i) a nucleic acid sequence encoding a nucleic acid-guided nuclease/reverse transcriptase fusion enzyme; (ii) a nucleic acid sequence encoding a first gRNA having a region of complementarity to a first sequence of an integration locus in a cell; (iii) a nucleic acid sequence encoding a second gRNA having a region of complementarity to a second sequence of the integration locus; (iv) a CF editing cassette comprising from 5′ to 3′: a nucleic acid sequence encoding a CFgRNA having a region of complementarity to a sequence of a target locus in the cell, and a nucleic acid sequence encoding a repair template; (v) a 5′ homology arm flanking a 5′ end of the CF editing cassette, the 5′ homology arm having homology to a third sequence of the integration locus; and (vi) a 3′ homology arm flanking a 3′ end of the CF editing cassette, the 3′ homology arm having homology to a fourth sequence of the integration locus.

In some aspects, the CFgRNA comprises from 5′ to 3′ a spacer region and a structural region recognized by the nucleic acid-guided nuclease/reverse transcriptase fusion enzyme.

In some aspects, the repair template comprises an edit and a primer binding site (PBS). In some aspects, the repair template further comprises a post-edit homology region. In some aspects, the repair template further comprises a nick-to-edit region.

In some aspects, the nucleic acid-guided nuclease/reverse transcriptase fusion enzyme comprises a nucleic acid-guided nickase and a reverse transcriptase. In some aspects, the nucleic acid-guided nickase comprises a MAD nickase or a variant thereof. In some aspects, the MAD nickase is selected from the group consisting of MADI, MAD2, MAD3, MAD4, MAD5, MAD6, MAD7R, MAD8, MAD9, MADIO, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299. In some aspects, the nucleic acid-guided nickase comprises a Cas nickase or a variant thereof. In some aspects, the nucleic acid-guided nickase comprises a Cas9 nickase or variant thereof. In some aspects, the nucleic acid-guided nickase comprises a Cpfl nickase or variant thereof.

In some aspects, the editing vector comprises a selectable marker. In some aspects, the CF editing cassette further comprises a selectable marker. In some aspects, the selectable marker is for selection and enrichment of cells having an integrated CF editing cassette. In some aspects, the selectable marker is an antibiotic resistance gene. In some aspects, the selectable marker is a puromycin resistance gene.

In some aspects, the editing vector further comprises self-targeting sequences having complementarity to the first gRNA and/or the second gRNA. In some aspects, the self-targeting sequences flank the CF editing cassette and the homology arms within the editing vector. In some aspects, the self-targeting sequences allow the integration of the CF editing cassette at the integration locus of the cellular genome.

In some aspects, integration locus is a safe harbor locus disposed centrally in an intergenic or intronic region of the cell. In some aspects, the integration locus is disposed within a coding region of the cell. In some aspects, the integration locus is disposed within a noncoding region of the cell.

In some aspects, CF editing cassette further comprises an edit to immunize the target locus and prevent re-nicking. In some aspects, the nucleic acid sequence encoding the repair template, or the repair template, comprises an edit to immunize the target locus and prevent re-nicking.

The present disclosure provides, in selected aspects, modules, instruments, and systems for automated multi-module cell processing for trackable nucleic acid-guided genome editing in multiple cells. Automated systems for cell processing that may be used for can be found, e.g., in U.S. Pat. Nos. 10,253,316; 10,329,559; 10,323,242; 10,421,959; 10,465,185; 10,519,437; 10,584,333; 10,584,334; 10,647,982; 10,689,645; 10,738,301; and 10,738,663.

In some aspects, the automated multi-module cell processing instruments of the present disclosure are designed for recursive genome editing, e.g., sequentially introducing multiple edits into genomes inside one or more cells of a cell population through two or more editing operations within the instruments.

In some aspects, the methods, compositions, modules, and instruments described herein may be utilized for efficient tracking of CF editing cassettes and/or CFgRNAs utilized during editing, for efficient tracking of ribonucleoprotein (RNP) based transfections, and for efficient tracking of non-plasmid based CFgRNA delivery via homologous recombination (HR) or non-homologous end joining (NHEJ) based integration of CF editing cassettes.

### Nucleic Acid-Guided Nickase/Reverse Transcriptase Fusion Protein Genome Editing, Generally

The compositions and methods described herein provide an alternative to traditional nucleic acid-guided nuclease editing (e.g., RNA-guided nuclease or CRISPR editing) used to introduce desired edits to a population of cells; that is, the compositions and methods described herein employ a nucleic acid-guided nickase/reverse transcriptase fusion enzyme (“nickase-RT fusion”) as opposed to a nucleic acid-guided nuclease (e.g., a “CRISPR nuclease”). The nickase-RT fusion employed herein differs from traditional CRISPR editing in that instead of initiating double-strand breaks in the target genome and homologous recombination to effect an edit, the nickase initiates a nick in a single strand of the target genome, e.g., the non-complementary strand.

Further, the fusion of the nickase to a reverse transcriptase, in combination with a CF editing cassette, eliminates the need for a donor DNA to be incorporated by homologous recombination. Instead, the CF editing cassette includes a nucleic acid sequence encoding a repair template—typically a ribonucleic acid—that serves as a template for the reverse transcription (“RT”) portion of the fusion enzyme to add the edit to the nicked strand at the target locus. That is, utilization of a nickase-RT fusion enables incorporation of the edit in the target genome by copying an RNA sequence (e.g., at the RNA level) rather than replacing a portion of the target locus with a donor DNA (e.g., at the DNA level).

The nickase—functioning as a single-strand cutter and having the specificity of a nucleic acid-guided nuclease—engages the target locus and nicks a strand of the target locus creating one or more free 3′ terminal nucleotides. The 3′ end of the repair template encoded by the CF editing cassette is then annealed to the nicked strand, and the reverse transcriptase utilizes the 3′ terminal nucleotide(s) of the nicked strand to copy the repair template of the CF editing cassette and create a “flap” containing the desired edit. Thereafter, endogenous repair mechanisms of the cells either repair the newly synthesized DNA by removing it and restoring the wild type sequence or removing the wild type flap and incorporating the edit. In summary, in certain aspects, the present methods and compositions are drawn to using the nickase-RT fusion to nick a strand of DNA at the target locus and using the CF editing cassette encoding the repair template to effect the desired edit on the strand via the reverse transcriptase portion of the nickase-RT fusion.

Generally, nucleic acid-guided nuclease editing typically begins with a nucleic acid-guided nuclease complexing with an appropriate guide nucleic acid in a cell which can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. For some nucleic acid-guided nucleases, two separate guide nucleic acid molecules that combine to function as a guide nucleic acid are used, e.g., a CRISPR RNA (crRNA) and trans-activating CRISPR RNA (tracrRNA). For other nucleic acid-guided nucleases, the guide nucleic acid may be a single guide nucleic acid that includes both the crRNA and tracrRNA sequences.

In general, a guide nucleic acid (e.g., gRNA or CFgRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and

RNA. In some aspects, a guide nucleic acid may comprise modified or non-naturally occurring nucleotides. In the present methods and compositions, the guide nucleic acid is RNA.

A guide nucleic acid comprises a guide sequence, where the guide sequence (as opposed to the scaffold sequence portion of the guide nucleic acid) is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 55%, 60%, 65%, 70%, 75%, 80%, 85%, 90%, 92.5%, 95%, 97.5%, 99%, or more. Optimal alignment may be determined with the use of any suitable algorithm for aligning sequences (e.g., without being limiting, BLAST™). In some aspects, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In some aspects of the present methods and compositions, the guide nucleic acids are provided as RNAs or sequences to be expressed from a plasmid or vector, and/or as sequences to be expressed from a CF editing cassette (e.g., CFgRNA) optionally inserted into a plasmid or vector, and comprise both the guide sequence and the scaffold sequence as a single transcript. The guide nucleic acids are engineered to target a desired target sequence by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) or a non-coding sequence (e.g., a regulatory polynucleotide, an intron, a PAM, or “junk” DNA).

As described above, in certain aspects, the guide nucleic acids may be part of CF editing cassettes that also encode for repair templates, which are used as templates for reverse transcription by the reverse transcriptase portion of the nickase-RT fusion. Each repair template generally comprises a desired edit to be incorporated into the target DNA sequence. Accordingly, the desired edit is integrated into the target DNA sequence via copying of the repair template by the nickase-RT fusion.

The target sequence is associated with a proto-spacer adjacent motif (PAM), which is a short nucleotide sequence recognized by the gRNA/nuclease complex. The precise preferred PAM sequence and length requirements for different nucleic acid-guided nucleases vary; however, PAMs typically are 2-8 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In certain aspects, the editing of a cellular target sequence both introduces a desired DNA change to the cellular target sequence, e.g., the genomic DNA of a cell, and removes, mutates, or renders inactive a PAM region or spacer region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing.

The range of target sequences that nucleic acid-guided nucleases can recognize is constrained by the need for a specific PAM to be located near the desired target sequence. As a result, it often can be difficult to target edits with the precision that is necessary for genome editing. It has been found that nucleases can recognize some PAMs very well (e.g., canonical PAMs), and other PAMs less well or poorly (e.g., non-canonical PAMs).

As for the nuclease or nickase-RT fusion component of the nucleic acid-guided nuclease editing system, a polynucleotide sequence encoding the nucleic acid-guided nuclease or nickase-RT fusion can be codon optimized for expression in particular cell types, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of nucleic acid-guided nuclease or nickase-RT fusion to be employed depends on many factors, such as what type of edit is to be made in the target sequence and whether an appropriate PAM is located close to the desired target sequence.

Nucleases of use in the methods described herein include but are not limited to nickases engineered from nucleic acid-guided nucleases such as Cas 9, Cas 12/Cpf1, MAD2, MAD2007, MAD2017, MAD2019, MAD297, MAD298, MAD299, MAD7®, or other MADZYME®, variants thereof, and nuclease or nickase fusions thereof. Nickase-RT fusion enzymes typically comprise one or more CRISPR nucleic acid-guided nucleases, each engineered to nick one DNA strand in the target DNA rather than making a double-stranded cut, and the nickase portion(s) are fused to a reverse transcriptase. In certain aspects of the present methods, the nickase-RT fusion nicks both strands of the target locus, albeit where the two nicks are staggered rather than at the same position which would result in a double-stranded cut. As with the guide nucleic acid, the nucleases or nickases may be encoded by one or more DNA sequences on a vector (e.g., an engine vector or an editing vector also comprising the CF editing cassette) and be under the control of a promoter—including inducible or constitutive promoters—or the nickase-RT fusion may be delivered as a protein or RNA-protein complex.

In addition to a nucleic acid sequence encoding the CFgRNA and a nucleic acid sequence encoding the repair template, a CF editing cassette or editing vector backbone may comprise one or more primer sites. The primer sites can be used to amplify the CF editing cassette or editing vector backbone by using oligonucleotide primers; for example, if the primer sites flank one or more of the other components of the CF editing cassette or editing vector backbone, e.g., the nucleic acid sequence encoding the CFgRNA and/or the nucleic acid sequence encoding the repair template.

Additionally, in some aspects, a vector encoding the nickase-RT fusion enzyme and/or the CF editing cassette further encodes one or more nuclear localization sequences (NLSs), such as about or more than about 1, 2, 3, 4, 5, 6, 7, 8, 9, 10, or more NLSs. In some aspects, the engineered nuclease comprises NLSs at or near the amino-terminus, NLSs at or near the carboxy-terminus, or a combination.

### Improved Nucleic Acid-Guided Nickase/Reverse Transcriptase Fusion Editing and Tracking of Edits

Creating a library of genomic edits requires tracking (e.g., identification) of editing events. Traditionally, in order to track editing events during one or more rounds of nucleic acid-guided nuclease editing, lentivector-based barcodes or episomal components are introduced into the host cells along with the editing guide nucleic acids, donor DNA, and/or nucleases for integration into the cell genomes. However, random integration of lentivector-based systems may adversely affect phonotype-genotype reagents, and episomes are inefficient and have low establishments rates, leading to a loss in library diversity. The present disclosure addresses the deficiencies of these and other trackable integration technologies.

In particular, the present disclosure provides compositions of matter, methods and instruments for nucleic acid-guided nickase/reverse transcriptase fusion (“nickase-RT fusion”) editing of live cells using CREATE fusion editing cassettes (e.g., “CF editing cassettes”) each encoding a gRNA (e.g., a “CFgRNA”) and a covalently linked repair template engineered to edit genomic DNA at a target locus and further integrate into the genomic DNA at a separate locus. The integration of the CF editing cassettes enables long-term, low level transcription of the CF editing cassette, thus facilitating tracking of corresponding nickase-RT fusion editing events on a one-to-one basis using single cell RNA sequencing methods, in addition to genomic DNA sequencing methods. Accordingly, each integrated CF editing cassette may serve as a proxy for one or more corresponding edits caused by that CF editing cassette. Utilizing the compositions and methods described herein, a single nickase-

RT fusion enzyme may be used to facilitate the incorporation of a desired edit into a cell genome at a first locus, and further facilitate the integration of the edit-causing and trackable CF editing cassette into the genome at a second locus. And, because the trackable feature integrated into the genome is the CF editing cassette, the CF editing cassette (e.g.encoding the CFgRNA and repair template) and/or other components of the nickase-RT fusion editing system do not need to be paired with a barcode, thus simplifying reagent manufacturing. Even further, a single integration locus, e.g., a safe harbor locus, once optimized, may enable consistent integration of trackable CF editing cassettes, thereby facilitating tracking of multiple editing events, e.g., during recursive editing.

FIG. 1A is a simplified block diagram of an example of a method 100 for editing live cells via nucleic acid-guided nickase/reverse transcriptase fusion (“nickase-RT fusion”) editing and for tracking the editing events. Looking at FIG. 1A, method 100 begins at 102 by designing and synthesizing CF editing cassettes encoding CFgRNAs and repair templates, which are designed to incorporate an edit into one or both DNA strands at a target locus, and to further integrate the CF editing cassette into the genome at an integration locus. That is, each CF editing cassette encodes a CFgRNA sequence and a covalently linked repair template sequence to be reverse transcribed comprising desired target genome edits, as well as a PAM and/or spacer mutation(s). Once the CF editing cassettes have been synthesized, the individual CF editing cassettes are amplified.

In addition, a nucleic acid-guided nickase/reverse transcriptase fusion (“nickase-RT fusion”) enzyme is designed 104. As described above, the nickase-RT fusion enzyme comprises, in order from amino terminus to carboxy terminus, or from carboxy terminus to amino terminus, a nucleic acid-guided nickase and a reverse transcriptase. The nickase-RT fusion enzyme may be delivered to the cells as a coding sequence in a vector (in some aspects under the control of an inducible promoter), such as the same or different vector as the CF editing cassette, or the nickase-RT fusion enzyme may be delivered to the cells as a protein or protein complex. In method 100, the nickase-RT fusion enzyme is delivered to the cells via a coding sequence in an editing vector further comprising a CF editing cassette.

At 106, a pair of additional gRNAs and a pair of homology arms are designed. The two gRNAs are designed to interact or complex with the nickase-RT fusion enzyme described above and bind to opposing strands of genomic DNA at an integration locus, thus facilitating the formation of a staggered double-stranded break (DSB) therein. Similarly, each of the homology arms is designed to have complementarity to a sequence or region of the integration locus at the staggered DSB. When assembled with the CF editing cassette such that they flank the CF editing cassette, the homology arms facilitate integration of the CF editing cassette into the genome at the break via HDR or other DNA repair pathways.

At 108, the CF editing cassette, the nickase-RT fusion enzyme, the pair of gRNAs targeting the integration locus, and/or the homology arms are assembled with vector backbones, such as plasmid backbones, to create editing vectors. In certain aspects, the CF editing cassette, the nickase-RT fusion enzyme, the gRNAs, and the homology arms are assembled together on a single editing vector. An example of an editing vector comprising all the aforementioned components is illustrated in FIG. 1C. In certain other aspects, however, the CF editing cassette and homology arms are assembled into an editing vector, and the nickase-RT fusion enzyme and gRNAs are assembled into a separate engine vector.

At 110, the engine and editing vectors are introduced into the live cells. A variety of delivery systems may be used to introduce (e.g., transform, transfect, or transduce) nucleic acid-guided nickase fusion editing system components into a host cell 110. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of particular interest is the use of electroporation, particularly flow-through electroporation (either as a stand-alone instrument or as a module in an automated multi-module system) as described in, e.g., U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; U.S. Pat. No. 10,738,663, issued 29 Sep. 2020; and U.S. Pat. No. 10,894,958, issued 19 Jan. 2021.

Once transformed 110, the next steps in method 100 include providing conditions for nucleic acid-guided nuclease editing 112 and for integration of the CF editing cassette into the genome 114. “Providing conditions” includes incubation of the cells in appropriate medium and may also include providing conditions to induce transcription of an inducible promoter (e.g., adding antibiotics, adding inducers, increasing temperature) for transcription of the CFgRNA and covalently linked repair template, the nickase-RT fusion, and/or the additional gRNAs. In certain aspects, the conditions for editing 112 and for genomic integration of the CF editing cassette 114 for subsequent tracking are the same and thus, these steps are performed simultaneously. In certain aspects, the conditions for editing 112 and for genomic integration of the CF editing cassette 114 are different (e.g., the additional gRNAs may be under the control of a different inducible promoter than other components of the editing system), and these steps may be performed either simultaneously or in sequence.

Once editing and integration is complete, the cells are allowed to recover and are preferably enriched for cells that have been edited and/or cells in which the CF editing cassette has integrated into the genome 116. Enrichment can be performed directly, such as via cells from the population that express a selectable marker, or by using surrogates, e.g., cell surface handles co-introduced with one or more components of the editing components. At this point in method 100, the cells can be characterized phenotypically or genotypically or, optionally, steps 102 to 114 or steps 110 to 114 may be repeated to make additional edits 118.

After recovery and enrichment of edited cells, the genomic DNA or RNA transcripts of the cells may be sequenced to track or analyze the editing events 120, where the integrated CF editing cassette(s) serve as accurate proxies for corresponding edits. For example, the cells may be lysed and DNA or RNA extracted, purified, amplified, prepared into libraries, and sequenced to track for integrated CF editing cassette(s). In certain aspects, genomic DNA is sequenced via any suitable high-throughput method, such as single molecule real time (SMRT) sequencing, nanopore sequencing, sequencing by synthesis (SBS) or Illumina sequencing. Ion Torrent sequencing, sequencing by ligation (SBL), combinatorial probe anchor synthesis (cPAS) sequencing, parallel pyrosequencing, microfluidic methods, etc. In certain aspects, the transcriptome of the cells is sequencing via any suitable high-throughput RNA sequencing (RNA-Seq) method.

FIG. 1B is a simplified graphic depiction of the mechanism of a nucleic acid-guided nickase enzyme/reverse transcriptase fusion enzyme edit. At left in FIG. 1B, a nickase-RT fusion enzyme and a CFgRNA of a CF editing cassette are shown bound to a target locus of the cell genome, where the target locus in the context of the methods and compositions herein is a locus of approximately 8 to 500 nucleotides in length, or 10 to 400 nucleotides in length, or 10 to 300 nucleotides in length. In one step, the nickase-RT fusion enzyme and the CFgRNA bind to the target locus and the nickase nicks a single DNA strand at the target locus, thus creating a 3′ “flap.” In order for the nickase-RT fusion enzyme and the CFgRNA to bind to the target locus and nick the genomic DNA, there must be a protospacer adjacent motif (PAM) appropriately located in or adjacent to the target locus and on the strand to be nicked and edited. The CFgRNA must also be complementary to a region of the strand to be edited and must include the desired edit to be incorporated.

At right in FIG. 1B shows the previously formed flap, where the reverse transcriptase (RT) portion of the nickase-RT fusion enzyme adds nucleotides to extend the 3′ free end of the nicked strand using the repair template of a CF editing cassette as a template, which includes the desired edit. The regions of the DNA strands that are synthesized by the RT may include a nick-to-edit region, an edit region, and a post-edit homology (PEH) region. The nick-to-edit region and the post-edit homology (PEH) region are complementary to the unedited (e.g., wildtype (wt)) strand, thus facilitating resolution of the edited flap with the unedited strand via endogenous repair mechanisms, e.g., homology-directed repair (HDR) or other repair pathways. The target locus may resolve into either wildtype, where the desired edit is not incorporated, or into an edited target locus. Once the DNA flap containing the edit is synthesized, an equilibrium is established between the newly synthesized 3′ flap and the wildtype 5′ flap. The equilibrium can be affected by the length of the edit, nick-to-edit distance, and/or post edit homology region. In order for the newly synthesized flap to be incorporated into the genome, the 5′ flap is likely degraded by an exonuclease or endonuclease. This allows the 3′ flap to anneal to the DNA, and a polymerase then likely fills in any missing nucleotides and a DNA ligase seals the nick.

At this stage, one DNA strand contains the edit while the second DNA strand does not. A mismatch repair or DNA replication process is likely responsible for copying the edit into both strands. Note that DNA replication and mismatch repair can also favor the wt strand as opposed to the edited strand. If the flap equilibration favors the wt 5′ flap, the newly synthesized flap is likely degraded and sealed in the same manner described above.

FIG. 1C schematically depicts an example of an editing vector layout according to aspects described herein. Note that the layout of the editing vector in FIG. 1C is only an example of, and does not limit aspects of, the present disclosure to any particular arrangement or orientation of components. As shown, the editing vector comprises a CF editing cassette comprising a nucleic acid sequence encoding a CFgRNA covalently linked to a repair template, a pair of homology arms flanking the CF editing cassette (e.g., a 5′ homology arm flanking the 5′ end of the cassette and a 3′ homology arm flanking the 3′ end of the cassette), a nucleic acid sequence encoding the nickase-RT fusion enzyme (“CFE”), and a pair of nucleic acid sequences encoding a pair of gRNAs (represented as “gRNA1” and “gRNA2”). Also shown in FIG. 1C are one or more promoters, which may be integrated into the editing vector to drive transcription of the CFgRNA and repair template, the nickase-RT fusion enzyme, and/or the gRNAs.

In certain aspects, the editing vector further includes a selectable marker, which may be arranged between the homology arms such that the selectable marker will integrate into the cell genome along with the CF editing cassette during integration events. Accordingly, the selectable marker may be used to “tag” and enrich for CF editing cassette integration events, and may also be under the transcriptional control of a promoter. In certain examples, selection for integration events with the selection marker may further upregulate editing at the target locus or other locus of the cell genome. Research has shown that selection for one integration event may upregulate editing at a separate, non-selected second site.

In certain aspects, as shown in FIG. 1C, the editing vector further includes one or more self-targeting or self-cutting sequences (e.g., spacer sequences), which may flank the homology arms. The self-targeting sequences may comprise regions of complementarity to the first gRNA and/or the second gRNA, or the self-targeting sequences may comprise regions of complementarity to other gRNAs utilized with the nucleic acid-guided nickase-RT fusion enzyme editing system, as well as a protospacer adjacent motif (PAM) site. Accordingly, during editing, the self-targeting sites may be targeted by corresponding gRNA-nickase complexes to induce one or more double stranded breaks in the editing vector around the CF editing cassette and flanking homology arms to facilitate removal and/or linearization of the CF editing cassette for integration into the integration locus.

FIG. 1D is a simplified depiction of nickase-RT fusion editing where the CF editing cassette used to edit the target locus is also integrated into the cell genome for tracking of editing events. As described above, the CFgRNA and repair template of the CF editing cassette are designed to associate or complex with the nickase-RT fusion and edit a target locus, while the gRNAs are designed to associate or complex with the nickase-RT fusion to create a staggered DSB (e.g., via gRNA-directed dual nicking) at an integration locus, shown at left in FIG. 1D. After the integration locus is nicked, the homology arms flanking the CF editing cassette (and selectable marker), which are designed to have complementarity to the integration locus at the staggered DSB, direct integration of the CF editing cassette at the staggered DSB site. Accordingly, a single nickase-RT fusion enzyme may be used to facilitate a desired edit and integrate the edit-causing CF editing cassette into the genome at a separate locus to facilitate tracking of editing events. And, because the trackable feature integrated into the genome is the CF editing cassette, the CFgRNA, repair template, and/or other components of the nickase-RT fusion editing system do not need to be paired with an additional barcode, thus simplifying the reagent manufacturing process. Further, a single integration locus, e.g., a safe harbor locus, once optimized, may enable consistent integration of trackable CF editing cassettes, thereby facilitating tracking of multiple editing events, e.g., during recursive editing.

FIG. 1E schematically depicts an example of an editing vector assembly according to aspects described herein. Note that the layout of the assembled editing vector in FIG. 1E is only an example of, and does not limit aspects of, the present disclosure to any particular arrangement or orientation of components. As shown, the editing vector comprises a CF editing cassette comprising, from a 5′ end to a 3′ end, a puromycin resistance gene (represented as “PuroR”) under the control of a PGK promoter, a poly A tail sequence, and a nucleic acid sequence encoding the CFgRNA and repair template (represented as “cassette”) under the control of a hU6 promoter for effecting an edit at a target locus. The editing vector further comprises a nickase-RT fusion enzyme (represented as “CFE”) linked to a pUC19 backbone, a 5′ homology arm flanking the 5′ end of the CF editing cassette and having complementarity to an integration locus, a 3′ homology arm flanking the 3′ end of the CF editing cassette and having complementarity to the integration locus, and a pair nucleic acid sequences encoding a pair of gRNAs (represented as “gRNA1” and “gRNA2”) under the control of hU6 promoters and configured to target the integration locus.

In the example of FIG. 1E, the integration locus-nicking gRNAs are each paired with their corresponding donor homology arm (each pair represented as “gBlocks”). Thus, in the example of FIG. 1E, the editing vector may be assembled, e.g., via thermal assembly, utilizing four components or pieces: the CF editing cassette encoding a CFgRNA and repair template, the nickase-RT fusion enzyme, and the two “gBlocks.” When preparing a library of edits or vectors, the gBlocks may be constants, or invariant components of each editing vector, with only the CF editing cassette and/or the nickase-RT fusion enzyme being variable.

FIG. 1F depicts the frequency (y-axis) of edited cells expressing blue fluorescent protein (“BFP+”). In FIG. 1F induced pluripotent stem cells (iPSC) expressing GFP (iPSC-GFP cells) are transfected with the editing vector of FIG. 1D where the “CFgRNA+Repair Template” targets a GFP-to-BFP edit, the selectable marker is a puromycin resistance gene, and gRNA1, gRNA2, and the homology arms target various integration loci (x-axis). The BFP+frequency is assessed before and after selection and enrichment of the edited cells via exposure to puromycin. Successful HDR-driven integration of the CF editing cassette results in the stable integration of the puromycin resistance marker.

### Automated Cell Editing Instruments and Modules to Perform Nucleic Acid-Guided Nuclease Editing in Cells

### Automated Cell Editing Instruments

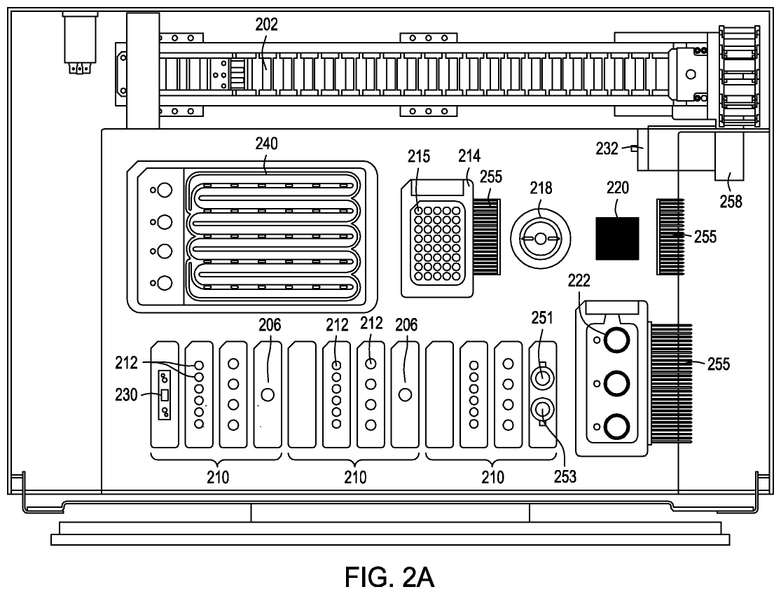

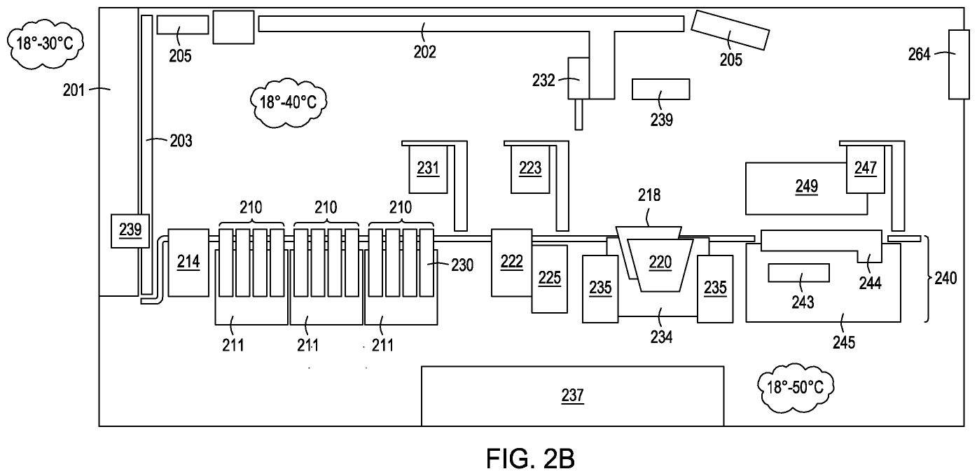

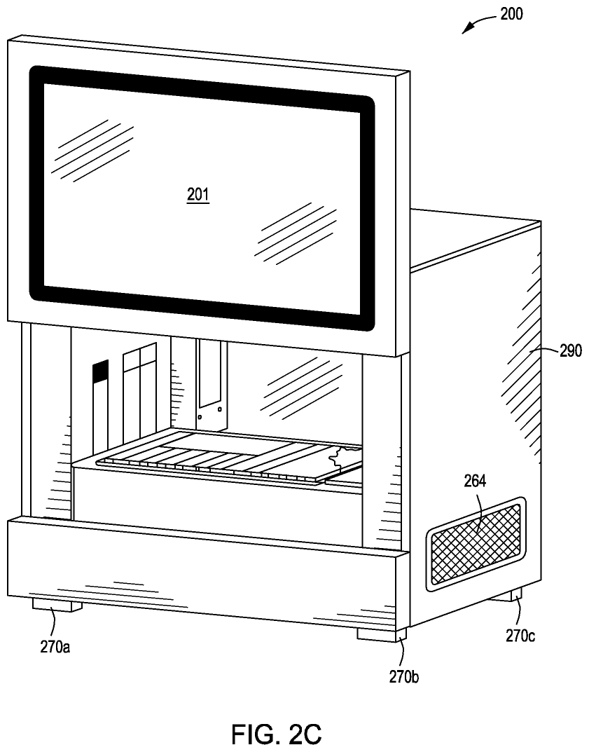

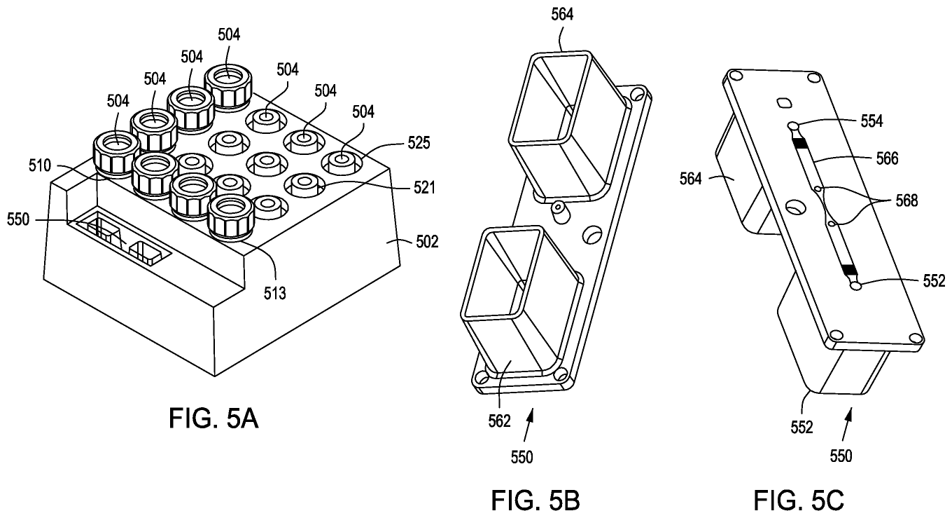

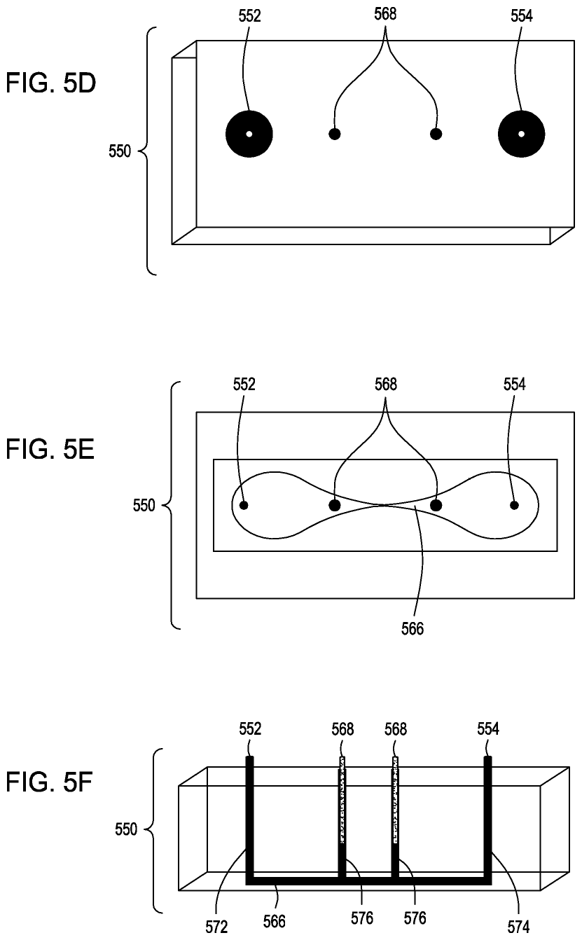

FIG. 2A depicts an example of an automated multi-module cell processing instrument 200 to, e.g., perform one of the exemplified novel methods using the novel nickase-RT fusion and CF editing cassette compositions described herein. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (e.g., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other aspects, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 5B-5F), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash cartridge 204 may be identical except for the consumables (reagents or other components contained within the various inserts) inserted therein.

In some implementations, the reagent cartridges 210) are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, NV (see, e.g., WO2018015544A1), or Beckman Coulter, Inc. of Fort Collins, CO. (see. e.g., US20160018427A1). Pipette tips may be provided in a pipette transfer tip supply (not shown) for use with the air displacement pipettor 232.

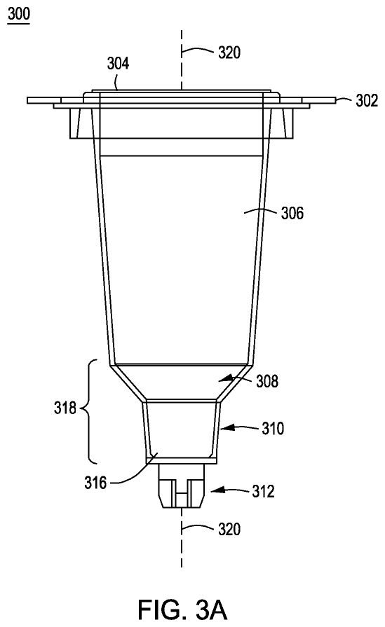

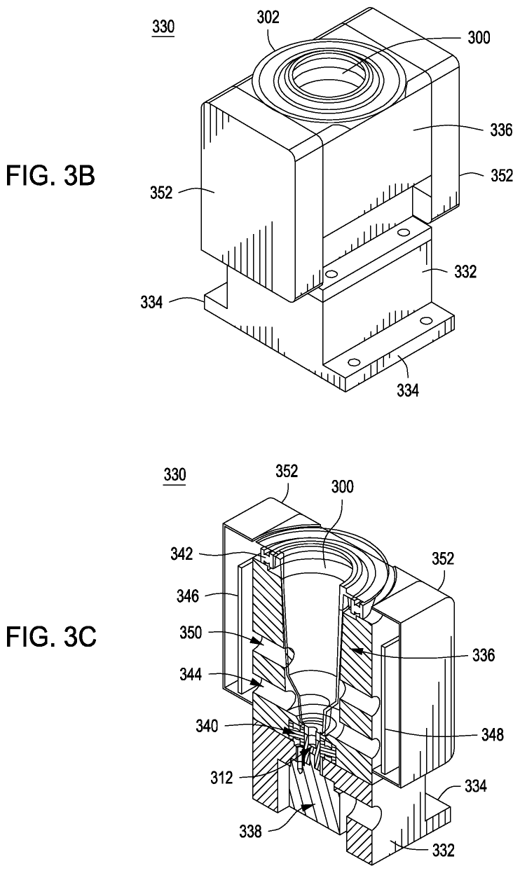

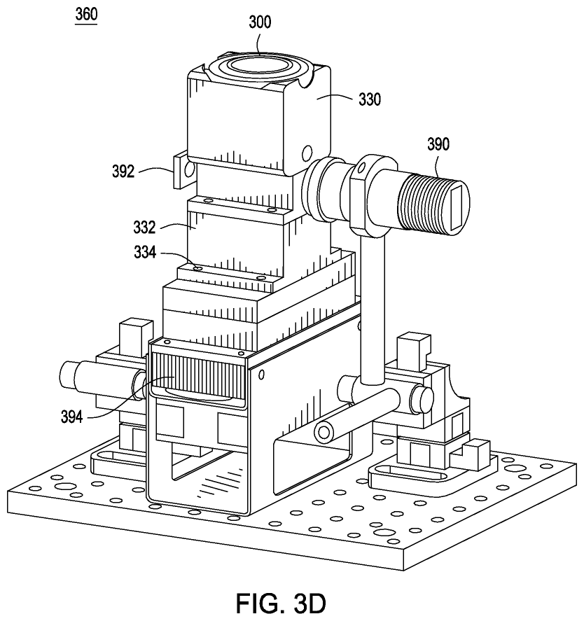

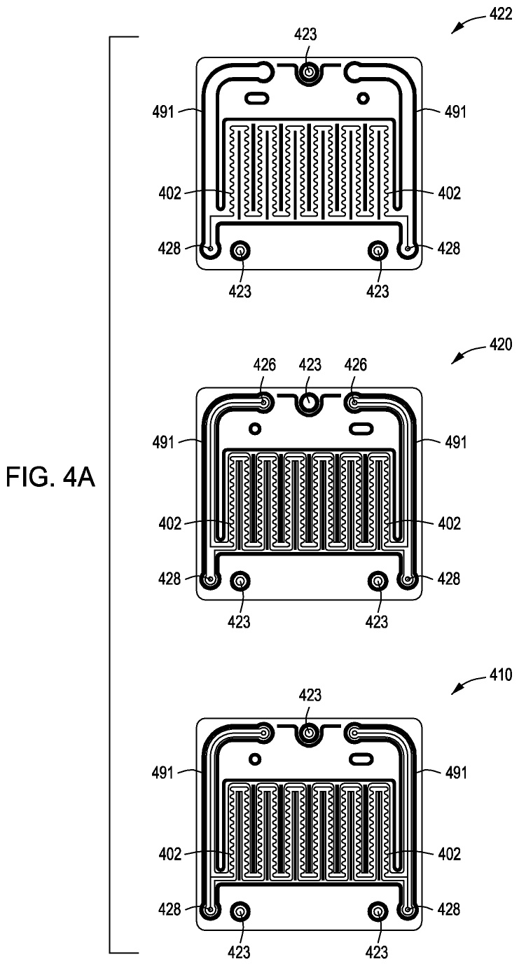

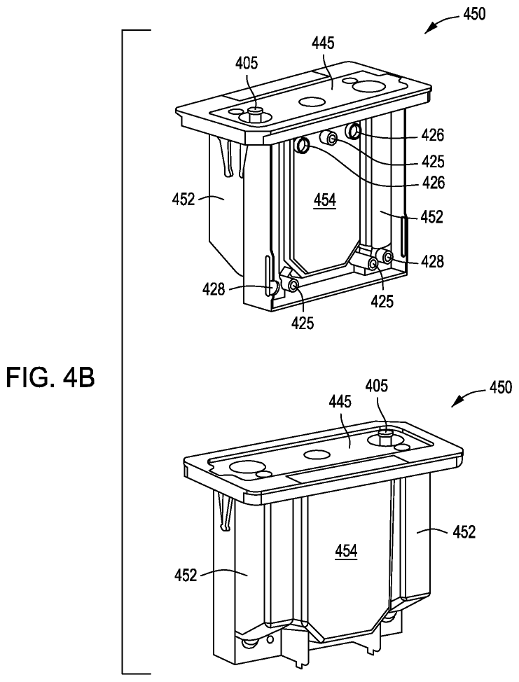

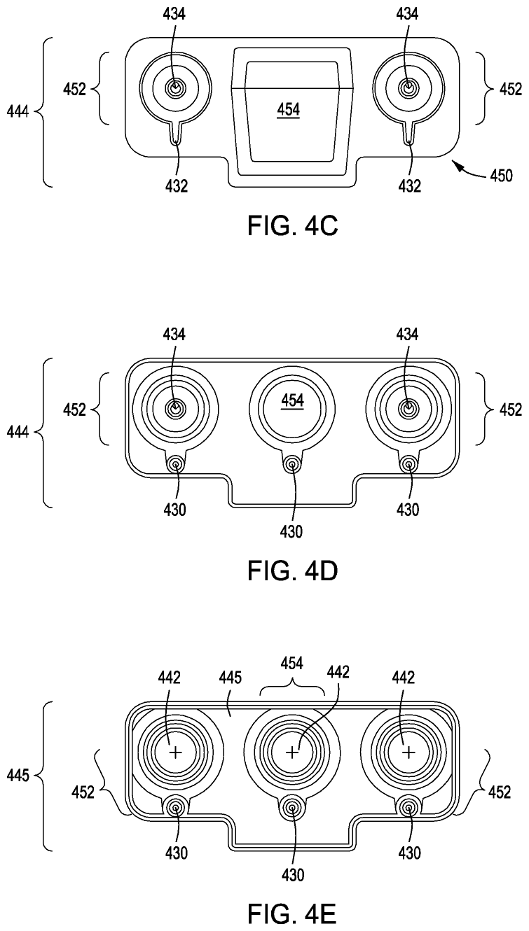

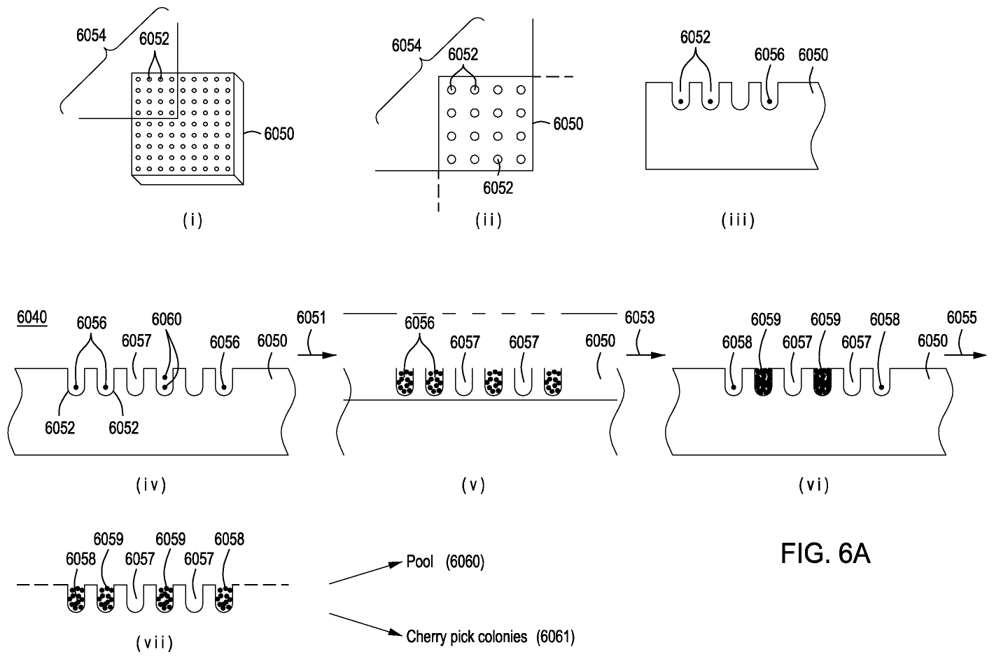

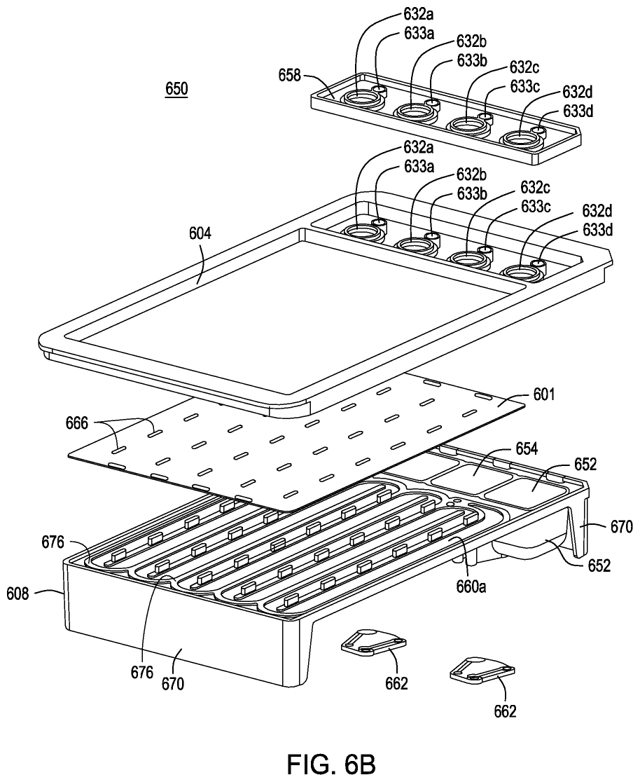

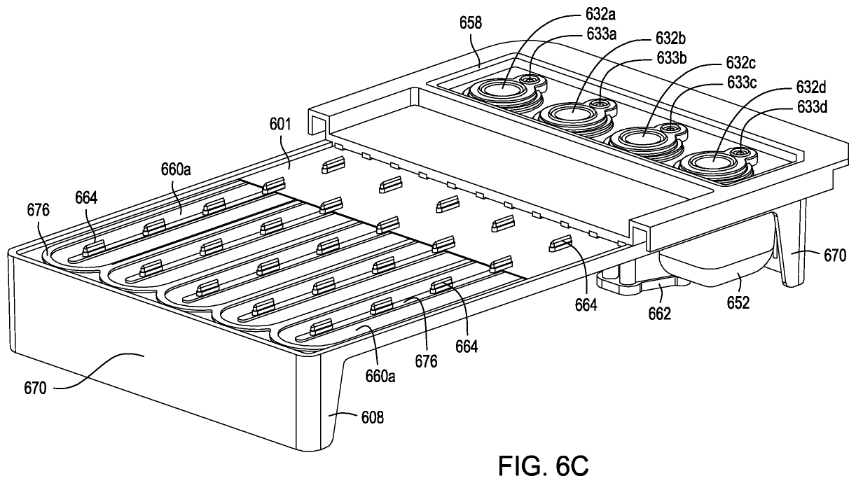

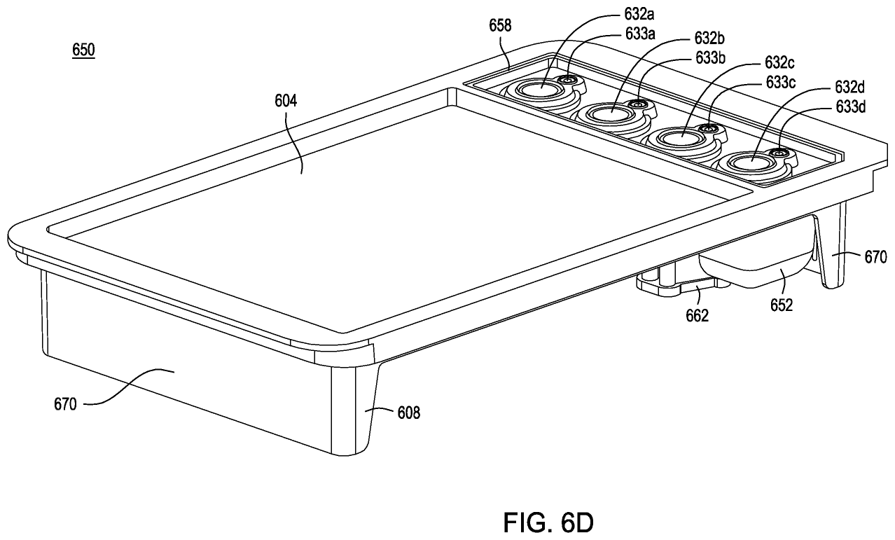

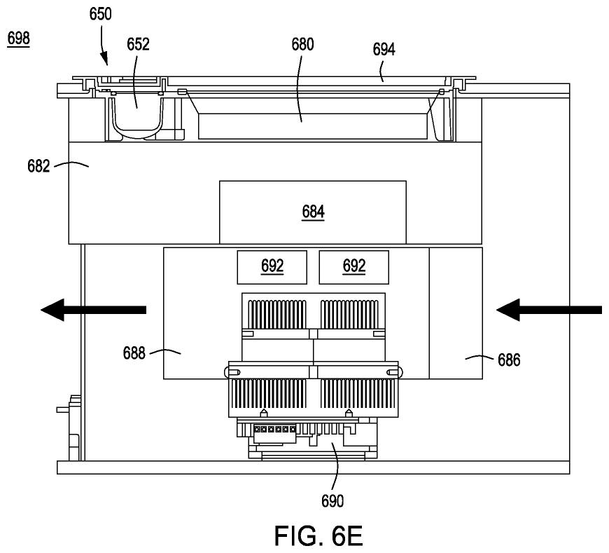

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the aspect illustrated in FIG. 2A, a cell growth module comprises a cell growth vial 218 (described in greater detail below in relation to FIGS. 3A-3D). Additionally seen is the TFF module 222 (described above in detail in relation to FIGS. 4A-4E). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6C-6F, served by, e.g., robotic liquid handing system 258 and air displacement pipettor 232. Additionally seen is a selection module 220. Also note the placement of three heatsinks 255.

FIG. 2B is a simplified representation of the contents of the example of a multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. Selection module is seen at 220. Also seen is the SWIIN module 240, comprising a SWIIN cartridge 241, where the SWIIN module also comprises a thermal assembly 245, illumination 243 (in this aspect, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIG. 2C illustrates a front perspective view of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this aspect, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. Nos. 10,253,310; 10,329,559; 10,323,242; 10, 421,959; 10,465,185; 10,519,437; 10,584,333; 10,584,334; 10,647,982; 10,689,645; 10,738,301; 10,738,663 and U.S. Ser. Nos. 16/412,175 and 16/988,694.

### The Rotating Cell Growth Module

FIG. 3A shows one aspect of a rotating growth vial 300 for use with the cell growth device and in the automated multi-module cell processing instruments described herein. The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some aspects of the rotating growth vial 300 have a second light path 308 in the tapered region of the tapered-to-constricted region 318. Both light paths in this aspect are positioned in a region of the rotating growth vial that is constantly filled with the cell culture (cells +growth media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some aspects, the motor drives the drive engagement mechanism 312 such that the rotating growth vial 300 is rotated in one direction only, and in other aspects, the rotating growth vial 300 is rotated in a first direction for a first amount of time or periodicity, rotated in a second direction (e.g., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another aspect, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some aspects, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 302 to overlap and engage with the cell growth device. In automated systems, the rotating growth vial 300 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 300 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 400. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material-or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly (methyl methacrylate (PMMA), polysulfone, polyurethane, and co-polymers of these and other polymers. Preferred materials include polypropylene, polycarbonate, or polystyrene. In some aspects, the rotating growth vial is inexpensively fabricated by, e.g., injection molding or extrusion.

FIG. 3B is a perspective view of one aspect of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail.

In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth via 316. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some aspects, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell

growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some aspects to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD (optical density), while the cell sample will deflect light rays and will have a lower percent transmittance and higher OD. As the cells grow in the media and become denser, transmittance will decrease and OD will increase. The processor (not shown) of the cell growth device 330—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth device 330 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this aspect, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one aspect, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the OD of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, ultraviolet (UV), or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 330 may include additional sensors for measuring, e.g., dissolved oxygen, carbon dioxide, pH, conductivity, and the like. For additional details regarding rotating growth vials and cell growth devices see U.S. Pat. No. 10,435,662, issued 8 Oct. 2019; U.S. Pat. No. 10,443,031, issued 15 Oct. 2019; and U.S. Ser. No. 16/552,981, filed 27 Aug. 2019 and U.S. Ser. No. 16/780,640, filed 3 Feb. 2020.

### The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in cell processing systems to perform the methods described herein is a module or component that can grow, perform buffer exchange, and/or concentrate cells and render them competent so that they may be transformed or transfected with the nucleic acids needed for engineering or editing the cell's genome.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422—specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this aspect mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple and tangential flow assembly 410 to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this aspect has a serpentine configuration and an undulating geometry, although other geometries may be used. Permeate member 420 also comprises countersinks 423, coincident with the countersinks 423 on retentate member 420.

On the left of FIG. 4A is a tangential flow assembly 410 comprising the retentate member 422 and permeate member 420 seen in this FIG. 4A. In this view; retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be between about 10 μm and 1000 μm wide, or between 200 μm and 800 μm wide, or between 300 μm and 700 μm wide, or between 400 μm and 600 μm wide; and between about 10 μm and 1000 μm high, or between 200 μm and 800 μm high, or between 300 μm and 700 μm high, or between 400 μm and 600 μm high. If the cross section of the flow channel 402 is generally round, oval or elliptical, the radius of the channel may be from between 50 μm and 1000 μm in hydraulic radius, or between 5 μm and 800 μm in hydraulic radius, or between 200 μm and 700 μm in hydraulic radius, or between 300 μm and 600 μm wide in hydraulic radius, or from between 200 and 500 μm in hydraulic radius. Moreover, the volume of the channel in the retentate 422 and permeate 420 members may be different depending on the depth of the channel in each member.

FIG. 4B shows front perspective (right) and rear perspective (left) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition gasket 445 is seen covering the top of reservoir assembly 450. Gasket 445 is described in detail in relation to FIG. 4E. At left in FIG. 4B is a rear perspective view of reservoir assembly 1250, where “rear” is the side of reservoir assembly 450 that is not coupled to the tangential flow assembly. Seen are retentate reservoirs 452, permeate reservoir 454, and gasket 445.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly (methyl methylacrylate) (PMMA), polysulfone, and polyurethane, and co-polymers of these and other polymers. If the TFF device/module is disposable, preferably it is made of plastic. In some aspects, the material used to fabricate the TFF device/module is thermally-conductive so that the cell culture may be heated or cooled to a desired temperature. In certain aspects, the TFF device is formed by precision mechanical machining, laser machining, electro discharge machining (for metal devices); wet or dry etching (for silicon devices); dry or wet etching, powder or sandblasting. photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIG. 4B and 4E depicts a gasket 445 that in operation is disposed on cover 444 of reservoir assemblies 450) shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again, at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configures to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall work flow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports 406, collecting the cell culture through a second retentate port 404 into a second retentate reservoir, optionally adding additional or different medium to the cell culture and optionally bubbling air or gas through the cell culture, then repeating the process, all while measuring, e.g., the optical density of the cell culture in the retentate reservoirs continuously or at desired intervals.

Measurements of optical densities at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log10) (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports 406. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420)) of the device. In this process, a fixed volume of cells in medium or buffer is driven through the device until the cell sample is collected into one of the retentate ports 404, and the medium/buffer that has passed through the membrane is collected through one or both of the permeate/filtrate ports 406. All types of prokaryotic and eukaryotic cells-both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells—except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen-or ECM-(extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports 404 while collecting the medium in one of the permeate/filtrate ports 406 is considered “one pass” of the cell sample. The transfer between retentate reservoirs “flips” the culture. The retentate and permeatee ports collecting the cells and medium, respectively, for a given pass reside on the same end of TFF device/module with fluidic connections arranged so that there are two distinct flow layers for the retentate and permeate/filtrate sides, but if the retentate port 404 resides on the retentate member of device/module (that is, the cells are driven through the channel above the membrane and the filtrate (medium) passes to the portion of the channel below the membrane), the permeate/filtrate port 406 will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port 404 and into the retentate reservoir (not shown). To initiate another “pass”, the cell sample is passed again through the TFF device, this time in a flow direction that is reversed from the first pass. The cell sample is collected by passing through the retentate port 404 and into retentate reservoir (not shown) on the opposite end of the device/module from the retentate port 404 that was used to collect cells during the first pass. Likewise, the medium/buffer that passes through the membrane on the second pass is collected through the permeate port 406 on the opposite end of the device/module from the permeate port 406 that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (e.g., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously. For further information and alternative aspects on TFFs see, e.g., U.S. Ser. Nos. 62/728,365, filed 7 Sep. 2018; 62/857,599, filed 5 Jun. 2019; and 62/867,415, filed 27 Jun. 2019.

### The Cell Transformation Module

FIG. 5A depicts an example of a combination reagent cartridge and electroporation device 500 (“cartridge”) that may be used in an automated multi-module cell processing instrument along with the TFF module. In addition, in certain aspects the material used to fabricate the cartridge is thermally-conductive, as in certain aspects the cartridge 500 contacts a thermal device (not shown), such as a Peltier device or thermoelectric cooler, that heats or cools reagents in the reagent reservoirs or reservoirs 504. Reagent reservoirs or reservoirs 504 may be reservoirs into which individual tubes of reagents are inserted as shown in FIG. 5A, or the reagent reservoirs may hold the reagents without inserted tubes. Additionally, the reservoirs in a reagent cartridge may be configured for any combination of tubes, co-joined tubes, and direct-fill of reagents.

In one aspect, the reagent reservoirs or reservoirs 504 of reagent cartridge 500 are configured to hold various size tubes, including, e.g., 250 mL tubes, 25 ml tubes, 10 mL tubes, 5 mL tubes, and Eppendorf or microcentrifuge tubes. In yet another aspect, all reservoirs may be configured to hold the same size tube, e.g., 5 mL tubes, and reservoir inserts may be used to accommodate smaller tubes in the reagent reservoir. In yet another aspect-particularly in an aspect where the reagent cartridge is disposable-the reagent reservoirs hold reagents without inserted tubes. In this disposable aspect, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one of ordinary skill in the art will appreciate given the present disclosure, the reagents contained in the reagent cartridge will vary depending on work flow; that is, the reagents will vary depending on the processes to which the cells are subjected in the automated multi-module cell processing instrument, e.g., protein production, cell transformation and culture, cell editing, etc.

Reagents such as cell samples, enzymes, buffers, nucleic acid vectors, expression cassettes, proteins or peptides, reaction components (such as, e.g., MgCl₂, dNTPs, nucleic acid assembly reagents, gap repair reagents, and the like), wash solutions, ethanol, and magnetic beads for nucleic acid purification and isolation, etc.

may be positioned in the reagent cartridge at a known position. In some aspects of cartridge 500, the cartridge comprises a script (not shown) readable by a processor (not shown) for dispensing the reagents. Also, the cartridge 500 as one component in an automated multi-module cell processing instrument may comprise a script specifying two, three, four, five, ten or more processes to be performed by the automated multi-module cell processing instrument. In certain aspects, the reagent cartridge is disposable and is pre-packaged with reagents tailored to performing specific cell processing protocols, e.g., genome editing or protein production. Because the reagent cartridge contents vary while components/modules of the automated multi-module cell processing instrument or system may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument, or, e.g., reagents for protein expression and a script that specifies the process steps for performing protein expression in an automated multi-module cell processing instrument.

For example, the reagent cartridge may comprise a script to pipette competent cells from a reservoir, transfer the cells to a transformation module, pipette a nucleic acid solution comprising a vector with expression cassette from another reservoir in the reagent cartridge, transfer the nucleic acid solution to the transformation module, initiate the transformation process for a specified time, then move the transformed cells to yet another reservoir in the reagent cassette or to another module such as a cell growth module in the automated multi-module cell processing instrument. In another example, the reagent cartridge may comprise a script to transfer a nucleic acid solution comprising a vector from a reservoir in the reagent cassette, nucleic acid solution comprising editing oligonucleotide cassettes in a reservoir in the reagent cassette, and a nucleic acid assembly mix from another reservoir to the nucleic acid assembly/desalting module, if present. The script may also specify process steps performed by other modules in the automated multi-module cell processing instrument.

For example, the script may specify that the nucleic acid assembly/desalting reservoir be heated to 50° C. for 30 minutes to generate an assembled product; and desalting and resuspension of the assembled product via magnetic bead-based nucleic acid purification involving a series of pipette transfers and mixing of magnetic beads, ethanol wash, and buffer.

As described in relation to FIGS. 5B and 5C below, the examples of reagent cartridges for use in the automated multi-module cell processing instruments may include one or more electroporation devices, preferably flow-through electroporation (FTEP) devices. In yet other aspects, the reagent cartridge is separate from the transformation module. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archaea, yeasts, other eukaryotic cells, bacteria, and other cell types. In some aspects, a cell is a prokaryotic cell. In some aspects, a cell is an archaea cell. In some aspects, a cell is a bacterial cell. In some aspects, a cell is an Escherichia coli cell. In some aspects, a cell is a eukaryotic cell. In some aspects, a cell is an animal cell. In some aspects, a cell is a mammalian cell. In some aspects, a cell is a human cell. In some aspects, the cell is an induced pluripotent stem cell (iPSC). In some aspects, a cell is a non-human animal cell. In some aspects, a cell is a non-human mammalian cell. In some aspects, a cell is a primate cell. In some aspects, a cell is a rodent cell. In some aspects, a cell is a plant cell. In some aspects, a cell is a fungal cell. In some aspects, a cell is a yeast cell. In some aspects, a cell is a Saccharomyces cerevisiae cell. In some aspects, a cell is a Schizosaccharomyces pombe cell.

Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif.) makes the GENE PULSER XCELLTM line of products to electroporate cells in cuvettes.

Traditionally, electroporation requires high field strength; however, the flow-through electroporation devices included in the reagent cartridges achieve high efficiency cell electroporation with low toxicity. The reagent cartridges of the disclosure allow for particularly easy integration with robotic liquid handling instrumentation that is typically used in automated instruments and systems such as air displacement pipettors.

Such automated instrumentation includes, but is not limited to, off-the-shelf automated liquid handling systems from Tecan (Mannedorf, Switzerland), Hamilton (Reno, NV), Beckman Coulter (Fort Collins, CO), etc.

FIGS. 5B and 5C are top perspective and bottom perspective views, respectively, of an example of an FTEP device 550 that may be part of (e.g., a component in) reagent cartridge 500 in FIG. 5A or may be a stand-alone module; that is, not a part of a reagent cartridge or other module. FIG. 5B depicts an FTEP device 550. The FTEP device 550 has wells that define cell sample inlets 552 and cell sample outlets 554. FIG. 5C is a bottom perspective view of the FTEP device 550 of FIG. 5B. An inlet well 552 and an outlet well 554 can be seen in this view. Also seen in FIG. 5C are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Pat. No. 10,435,713, issued 8 Oct. 2019; U.S. Pat. No. 10,443,074, issued 15 Oct. 2019; U.S. Pat. No. 10,323,258, issued 18 Jun. 2019; U.S. Pat. No. 10,508,288, issued 17 Dec. 2019; U.S. Pat. No. 10,415,058, issued 17 Sep. 2019; and U.S. Ser. No. 16/550,790, filed 26 Aug. 2019; and U.S. Ser. No. 16/571,080, filed 14 Sep. 2019. Further, other aspects of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889, issued 13 Aug. 2019; U.S. Pat. No. 10,406,525, issued 10 Sep. 2019; U.S. Pat. No. 10,478,822, issued 19 Nov. 2019; U.S. Pat. No. 10,576,474, issued 3 Feb. 2020; and U.S. Ser. No. 16/749,757, filed 22 Jan. 2020.

Additional details of the FTEP devices are illustrated in FIGS. 5D-5F. Note that in the FTEP devices in FIGS. 5D-5F the electrodes are placed such that a first electrode is placed between an inlet and a narrowed region of the flow channel, and the second electrode is placed between the narrowed region of the flow channel and an outlet. FIG. 5D shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device. FIG. 5E shows a cutaway view from the top of the FTEP device 550, with the inlet 552, outlet 554, and electrodes 568 positioned with respect to a flow channel 566.

FIG. 5F shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 576 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining. The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain aspects, the FTEP devices are reusable and, in some aspects, the FTEP devices are disposable. In additional aspects, the FTEP devices may be autoclavable.

The electrodes 508 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (e.g., non-disposable) flow-through FTEP device is desired-as opposed to a disposable, one-use flow-through FTEP device-the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be between 3 cm and 15 cm in length, or between 4 cm and 12 cm in length, or between 4.5 cm and 10 cm in length. The overall width of the FTEP device may be between 0.5 cm and 5 cm, or between 0.75 cm and 3 cm, or between 1 cm and 2.5 cm, or between 1 cm and 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In aspects of the FTEP device where reservoirs are used to introduce cells and exogenous material into the FTEP device, the reservoirs range in volume from between 100 μL and 10 mL, or between 500 μL and 75 mL, or between 1 and to 5 mL.

The flow rate in the FTEP ranges from between 0.1 mL and 5 mL per minute, or between 0.5 mL and 3 mL per minute, or between 1.0 mL and 2.5 mL per minute. The pressure in the FTEP device ranges from between 1 and 30 PSI, between 2 and 10 PSI, or between 3 and 5 PSI.

To avoid different field intensities between the electrodes, the electrodes should be arranged in parallel. Furthermore, the surface of the electrodes should be as smooth as possible without pin holes or peaks. Electrodes having a roughness Rz of between 1 μm and 10 μm are preferred. In another aspect of the invention, the flow-through electroporation device comprises at least one additional electrode which applies a ground potential to the FTEP device.

### Cell Singulation and Enrichment Device

FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this aspect, Poisson or substantial Poisson loading has taken place; that is, each microwell has one or no cells, and the likelihood that any one microwell has more than one cell is low. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then editing is allowed to occur 6053.

After editing 6053, many cells in the colonies of cells that have been edited die as a result of the nicks caused by active editing or by fitness effects from the edits themselves and there is a lag in growth for the edited cells that do survive but must repair and recover following editing (microwells 6058), where cells that do not undergo editing thrive (microwells 6059) (vi). All cells are allowed to continue grow to establish colonies and normalize, where the colonies of edited cells in microwells 6058 catch up in size and/or cell number with the cells in microwells 6059 that do not undergo editing (vii). Once the cell colonies are normalized, either pooling 6060 of all cells in the microwells can take place, in which case the cells are enriched for edited cells by eliminating the bias from non-editing cells and fitness effects from editing; alternatively, colony growth in the microwells is monitored after editing, and slow growing colonies (e.g., the cells in microwells 6058) are identified and selected 6061 (e.g., “cherry picked”) resulting in even greater enrichment of edited cells.

In growing the cells, the medium used will depend on the type of cells being edited—e.g., bacterial, yeast or mammalian. For example, medium for yeast cell growth includes LB, SOC, TPD, YPG, YPAD, MEM and DMEM.

A module useful for performing the method depicted in FIG. 6A is a solid wall isolation, incubation, and normalization (SWIIN) module. FIG. 6B depicts an aspect of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6B comprises from the top down, a reservoir gasket or cover 658, a retentate member 604 (where a retentate flow channel cannot be seen in this FIG. 6B), a perforated member 601 swaged with a filter (filter not seen in FIG. 6B), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 can be seen disposed on the top of permeate member 608 as well. The perforations that form the wells on perforated member 601 are not seen in this FIG. 6B; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6B).

In this FIG. 6B, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this aspect of a SWIIN module, the perforated member includes through-holes to accommodate ultrasonic tabs disposed on the permeate member. Thus, in this aspect the perforated member is fabricated from 316 stainless steel, and the perforations form the walls of microwells while a filter or membrane is used to form the bottom of the microwells. Typically, the perforations (microwells) are approximately 150 μm to 200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nL, the volume of the microwells may be between 1 nL and 25 nL, or preferably between 2 nL and 10 nL, and even more preferably between 2 nL and 4 nL. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 um, however for other cell types (e.g., such as for mammalian cells), the pore sizes can be as high as from 10.0 μm to 20.0 μm, or more. Indeed, the pore sizes useful in the cell concentration device/module include filters with sizes from 0.10 μm. 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), poly tetrafluoroethylene (PTFE), nylon, or glass fiber.

The cross-section configuration of the mated serpentine channel may be

round, elliptical, oval, square, rectangular, trapezoidal, or irregular. If square, rectangular, or another shape with generally straight sides, the cross section may be between about 2 mm and 15 mm wide, between 3 mm and 12 mm wide, or between 5 mm and 10 mm wide. If the cross section of the mated serpentine channel is generally round, oval or elliptical, the radius of the channel may be between about3 mm and 20 mm in hydraulic radius, between 5 mm and 15 mm in hydraulic radius, or between 8 mm and 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, from about 4 mL to 60 mL, from about 5 mL to about 40 mL, or from about 6 mL to about 20 mL (note these volumes apply to a SWIIN module comprising a, e.g., 50-500K perforation member).

The volume of the reservoirs may range between 5 mL and 50 mL, or between 7 mL and 40 mL, or between 8 mL and 30 mL or between 10 mL and 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other aspects, the retentate and permeate members can be between 75 mm and 400 mm in length, between 100 mm and 300 mm in length, or between 150 mm and 250 mm in length; between 50 mm and 250 mm in width, between 75 mm and 200 mm in width, or between 100 mm and 150 mm in width; and between 2 mm and 15 mm in thickness, between 4 mm and 10 mm in thickness, or between 5 mm and 8 mm in thickness. In some aspects, the retentate (and permeate) members may be fabricated from PMMA (poly (methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see. e.g., FIG. 6E and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, CA)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, MA) (also see Levin-Reisman, et al., Nature Methods, 7:737-39 (2010)). Cell growth for, e.g., mammalian cells may be monitored by, e.g., the growth monitor sold by IncuCyte (Ann Arbor, MI) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, NJ); Molecular Devices (QPix 400® system, San Jose, CA); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See. e.g., FIG. 6E and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through permeate ports 611. The medium flows upward through filter 603 to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter 603 and pooled. Alternatively, if cherry picking is desired, the growth of the cell colonies in the microwells is monitored, and slow-growing colonies are directly selected; or, fast-growing colonies are eliminated.

FIG. 6C is a top perspective view of a SWIIN module with the retentate and perforated members in partial cross section. In this FIG. 6C, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6C) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6C) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes 666 of perforated member 601. There is also a support 670 at the end distal reservoirs 652, 654 of permeate member 608.

FIG. 6D is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9) pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6E depicts the aspect of the SWIIN module in FIGS. 6B-6D further comprising a heat management system including a heater and a heated cover. The heater cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6E, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see U.S. Ser. No. 16/399,988, filed 30 Apr. 2019; U.S. Ser. No. 16/454,865, filed 26 Jun. 2019; and U.S. Ser. No. 16/540,606, filed 14 Aug. 2019. For alternative isolation, incubation and normalization modules, see U.S. Ser. No. 16/536,049, filed 8 Aug. 2019.

### Use of the Automated Multi-Module Cell Processing Instrument

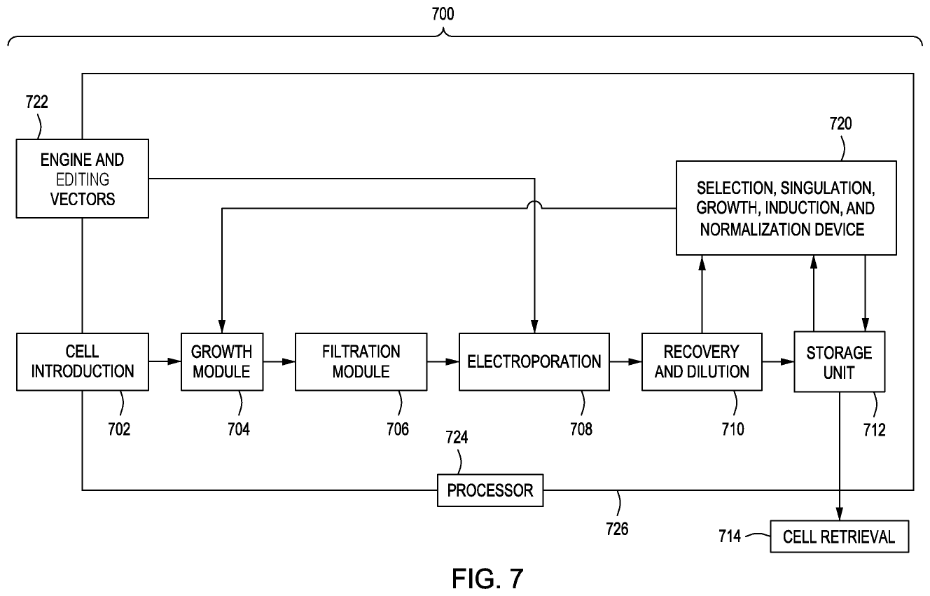

FIG. 7 illustrates an aspect of a multi-module cell processing instrument. This aspect depicts an example of a system that performs recursive and trackable nickase-RT fusion editing on a cell population. The cell processing instrument 700 may include a housing 726, a reservoir for storing cells to be transformed or transfected 702, and a cell growth module (comprising, e.g., a rotating growth vial) 704. The cells to be transformed are transferred from a reservoir 702 to the cell growth module 704 to be cultured until the cells hit a target OD. Once the cells hit the target OD, the growth module may cool or freeze the cells for later processing or transfer the cells to a cell concentration (e.g., filtration) module 706 where the cells are subjected to buffer exchange and rendered electrocompetent and the volume of the cells may be reduced substantially. Once the cells have been concentrated to an appropriate volume, the cells are transferred to electroporation device 708 or other transformation module. In addition to the reservoir for storing cells 702, the multi-module cell processing instrument includes a reservoir for storing the engine and editing vectors or engine +editing vectors or vectors and proteins to be introduced into the electrocompetent cell population 722. The vector backbones and editing cassettes are transferred to the electroporation device 708, which already contains the cell culture grown to a target OD. In the electroporation device 708, the nucleic acids (or nucleic acids and proteins) are electroporated into the cells. Following electroporation, the cells are transferred into an optional recovery and dilution module 710, where the cells recover briefly post-transformation.

After recovery, the cells may be transferred to a storage module 712, where the cells can be stored at, e.g., 4° C. or −20° C. for later processing, or the cells may be diluted and transferred to a selection/singulation/growth/induction/editing/normalization (SWIIN) module 720. In the SWIIN 720, the cells are arrayed such that there is an average of one to twenty or fifty or so cells per microwell. The arrayed cells may be in selection medium to select for cells that have been transformed or transfected with the editing vector(s). Once singulated, the cells grow through 2 to 50 doublings and establish colonies. Once colonies are established, editing is induced by providing conditions (e.g., temperature, addition of an inducing or repressing chemical) to induce editing. Editing is then initiated and allowed to proceed, the cells are allowed to grow to terminal size (e.g., normalization of the colonies) in the microwells and then are treated to conditions that cure the editing vector from this round. Once cured, the cells can be flushed out of the microwells and pooled, then transferred to the storage (or recovery) unit 712 or can be transferred back to the growth module 704 for another round of editing. In between pooling and transfer to a growth module, there typically is one or more additional steps, such as cell recovery, medium exchange (rendering the cells electrocompetent), cell concentration (typically concurrently with medium exchange by, e.g., filtration.

Note that the selection/singulation/growth/induction/editing/normalization and curing modules may be the same module, where all processes are performed in, e.g., a solid wall device, or selection and/or dilution may take place in a separate vessel before the cells are transferred to the solid wall singulation/growth/induction/editing/normalization/editing module (SWIIN). Similarly, the cells may be pooled after normalization, transferred to a separate vessel, and cured in the separate vessel. Once the putatively-edited cells are pooled, they may be subjected to another round of editing, beginning with growth, cell concentration and treatment to render electrocompetent, and transformation by yet another donor nucleic acid in another editing cassette via the electroporation module 708.

In electroporation device 708, the cells selected from the first round of editing are transformed by a second set of editing vectors and the cycle is repeated until the cells have been transformed and edited by a desired number of, e.g., CF editing cassettes. The multi-module cell processing instrument exemplified in FIG. 7 is controlled by a processor 724 configured to operate the instrument based on user input or is controlled by one or more scripts including at least one script associated with the reagent cartridge. The processor 724 may control the timing, duration, and temperature of various processes, the dispensing of reagents, and other operations of the various modules of the instrument 700. For example, a script or the processor may control the dispensing of cells, reagents, vectors, and editing oligonucleotides; which editing oligonucleotides are used for cell editing and in what order: the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 7, then the resulting edited culture may go through another (or several or many) rounds of additional editing (e.g., recursive editing) with different editing cassettes (or ribozyme-containing editing cassettes). For example, the cells from round 1 of editing may be diluted and an aliquot of the edited cells edited by editing cassette A may be combined with editing cassette B, an aliquot of the edited cells edited by editing cassette A may be combined with editing cassette C, an aliquot of the edited cells edited by editing cassette A may be combined with editing cassette D, and so on for a second round of editing. After round two, an aliquot of each of the double-edited cells may be subjected to a third round of editing, where, e.g., aliquots of each of the AB-, AC-, AD-edited cells are combined with additional editing cassettes, such as editing cassettes X, Y, and Z. That is, double-edited cells AB may be combined with and edited by editing cassettes X, Y, and Z to produce triple-edited edited cells ABX, ABY, and ABZ; double-edited cells AC may be combined with and edited by editing cassettes X, Y, and Z to produce triple-edited cells ACX, ACY, and ACZ; and double-edited cells AD may be combined with and edited by editing cassettes X, Y, and Z to produce triple-edited cells ADX, ADY, and ADZ, and so on. In this process, many permutations and combinations of edits can be executed, leading to very diverse cell populations and cell libraries.

In any recursive process, it is advantageous to “cure” the editing vectors comprising the CF editing cassette. “Curing” is a process in which one or more editing vectors used in the prior round of editing is eliminated from the transformed cells. (See, e.g., curing can be accomplished by, e.g., cleaving the editing vector(s) using a curing plasmid thereby rendering the editing vectors nonfunctional; diluting the editing vector(s) in the cell population via cell growth (that is, the more growth cycles the cells go through, the fewer daughter cells will retain the editing vector(s)), or by, e.g., utilizing a heat-sensitive origin of replication on the editing vector. The conditions for curing will depend on the mechanism used for curing; that is, in this example, how the curing plasmid cleaves the editing vector.

A variety of further modifications and improvements in and to the compositions, methods, and modified cells of the present disclosure will be apparent to those skilled in the art. The following non-limiting, embodiments are specifically envisioned:

1. A method for performing nucleic acid-guided nuclease/reverse transcriptase fusion editing in a genome of a live cell, comprising:
(a) providing the live cell, wherein the live cell comprises a target locus and an integration locus;
(b) providing a nucleic acid-guided nuclease/reverse transcriptase fusion enzyme;
(c) providing a first guide RNA (gRNA) having a region of complementarity to a first sequence of the integration locus;
(d) providing a second gRNA having a region of complementarity to a second sequence of the integration locus;
(e) providing an editing vector, the editing vector comprising:
   (i) a CF editing cassette comprising from 5′ to 3′:
      (A) a nucleic acid sequence encoding a CFgRNA having a region of complementarity to a sequence of the target locus, and
   (B) a nucleic acid sequence encoding a repair template5′3′;
   (ii) a 5′ homology arm flanking a 5′ end of the CF editing cassette, the 5′ homology arm having homology to a third sequence of the integration locus; and (iii) a 3′ homology arm flanking a 3′ end of the CF editing cassette, the 3′ homology arm having homology to a fourth sequence of the integration locus;
(f) providing conditions to allow the nucleic acid-guided nuclease/reverse transcriptase fusion enzyme, the CFgRNA, and the repair template to bind to the target locus;
(g) allowing the nucleic acid-guided nuclease/reverse transcriptase fusion enzyme, the CFgRNA, and the repair template to edit the target locus;
(h) providing conditions to allow the nucleic acid-guided nuclease/reverse transcriptase fusion enzyme and the first and second gRNAs to bind and nick at the integration locus; and
(i) allowing the CF editing cassette to integrate into the integration locus.
   2. The method of embodiment 1, wherein the CFgRNA comprises from 5′ to 3′ a spacer region and a structural region recognized by the nucleic acid-guided nuclease/reverse transcriptase fusion enzyme.
   3. The method of embodiment 1 or 2, wherein the repair template comprises an edit and a primer binding site (PBS).
   4. The method of embodiment 3, wherein the repair template further comprises a post-edit homology region.
   5. The method of embodiment 3 or 4, wherein the repair template further comprises a nick-to-edit region.
   6. The method of embodiment 1, further comprising:

sequencing the genome or a transcriptome of the cell to track for integration of the CF editing cassette, the integration of the CF editing cassette representing a nucleic acid-guided nickase/reverse transcriptase fusion editing event.

7. The method of any one of embodiments 1 to 6, wherein the nucleic acid-guided nuclease/reverse transcriptase fusion enzyme comprises a nucleic acid-guided nickase and a reverse transcriptase.
8. The method of embodiment 7, wherein the nucleic acid-guided nickase comprises a MAD nickase or a variant thereof.
9. The method of embodiment 7, wherein the nucleic acid-guided nickase comprises a Cas nickase or a variant thereof.
10. The method of any one of embodiments 1 to 9, wherein the editing vector further comprises a nucleic acid sequence encoding the nucleic acid-guided nuclease/reverse transcriptase fusion enzyme.
11. The method of any one of embodiments Ito 10, wherein the editing vector further comprises a nucleic acid sequence encoding the first gRNA and a nucleic acid sequence encoding the second gRNA.
12. The method of any one of embodiments 1 to 9, further comprising providing an engine vector comprising a nucleic acid sequence encoding the nucleic acid-guided nuclease/reverse transcriptase fusion enzyme, wherein the engine vector is different from the editing vector.
13. The method of embodiment 12, wherein the engine vector further comprises a nucleic acid sequence encoding the first gRNA and a nucleic acid sequence encoding the second gRNA.
14. The method of any one of embodiments Ito 13, wherein the CF editing cassette further comprises a selectable marker.
15. The method of embodiment 14, wherein the selectable marker is for selection and enrichment of cells having an integrated CF editing cassette.
16. The method of embodiment 14 or 15, further comprising selecting and enriching for cells having an integrated CF editing cassette.
17. The method of any one of embodiments 14 to 16, wherein the selectable marker is a puromycin resistance gene.
18. The method of any one of embodiments 1 to 17, wherein the editing vector further comprises self-targeting sequences having complementarity to the first gRNA and/or the second gRNA.
19. The method of any one of embodiments 1 to 18, wherein the integration locus is a safe harbor locus disposed centrally in an intergenic or intronic region of the cell.
20. The method of any one of embodiments 1 to 18, wherein the integration locus is disposed within a coding region of the cell.
21. The method of any one of embodiments Ito 18, wherein the integration locus is disposed within a noncoding region of the cell.
22. The method of any one of embodiments 1 to 21, wherein the CF editing cassette further comprises an edit to immunize the target locus and prevent re-nicking.
23. An editing system comprising one or more vectors comprising:
a nucleic acid sequence encoding a nucleic acid-guided nuclease/reverse transcriptase fusion enzyme;
a nucleic acid sequence encoding a first gRNA having a region of complementarity to a first sequence of an integration locus in a cell;
a nucleic acid sequence encoding a second gRNA having a region of complementarity to a second sequence of the integration locus; a CF editing cassette comprising from 5′ to 3′;
   a nucleic acid sequence encoding a CFgRNA having a region of complementarity to a sequence of a target locus in the cell, and
   a nucleic acid sequence encoding a repair template;
a 5′ homology arm flanking a 5′ end of the CF editing cassette, the 5′ homology arm having homology to a third sequence of the integration locus; and
a 3′ homology arm flanking a 3′ end of the CF editing cassette, the 3′ homology arm having homology to a fourth sequence of the integration locus.
   24. The editing system of embodiment 23, wherein the CFgRNA comprises from 5′ to 3′ a spacer region and a structural region recognized by the nucleic acid-guided nuclease/reverse transcriptase fusion enzyme.
   25. The editing system of embodiment 23 or 24, wherein the repair template comprises an edit and a primer binding site (PBS).
   26. The editing system of embodiment 25, wherein the repair template further comprises post-edit homology region.
   27. The editing system of embodiment 25 or 26, wherein the repair template further comprises a nick-to-edit region.
   28. The editing system of any one of embodiments 23 to 27, wherein the nucleic acid-guided nuclease/reverse transcriptase fusion enzyme comprises a nucleic acid-guided nickase and a reverse transcriptase.
   29. The editing system of embodiment 28, wherein the nucleic acid-guided nickase comprises a MAD nickase or a variant thereof.
   30. The editing system of embodiment 28, wherein the nucleic acid-guided nickase comprises a Cas nickase or a variant thereof.
   31. The editing system of any one of embodiments 23 to 30, wherein the one or more vectors comprise an editing vector, and wherein the editing vector comprises the CF editing cassette, the 5′ homology arm, and the 3′ homology arm.
   32. The editing system of embodiment 31, wherein the editing vector further comprises a nucleic acid sequence encoding the nucleic acid-guided nuclease/reverse transcriptase fusion enzyme.
   33. The editing system of embodiment 31 or 32, wherein the editing vector further comprises a nucleic acid sequence encoding the first gRNA and a nucleic acid sequence encoding the second gRNA.
   34. The editing system of any one of embodiments 31 to 33, wherein the editing vector further comprises self-targeting sequences having complementarity to the first gRNA and/or the second gRNA.
   35. The editing system of any one of embodiments 23 to 30, wherein the one or more vectors comprise an engine vector, and wherein the engine vector comprises a nucleic acid sequence encoding the nucleic acid-guided nuclease/reverse transcriptase fusion enzyme.
   36. The editing system of embodiment 35, wherein the engine vector further comprises a nucleic acid sequence encoding the first gRNA and a nucleic acid sequence encoding the second gRNA.
   37. The editing system of any one of embodiments 23 to 36, wherein the CF editing cassette further comprises a selectable marker.
   38. The editing system of embodiment 37, wherein the selectable marker is a puromycin resistance gene.
   39. The editing system of embodiment 37 or 38, wherein the selectable marker is for selection and enrichment of cells having an integrated CF editing cassette.
   40. The editing system of any one of embodiments 23 to 39, wherein the integration locus is a safe harbor locus disposed centrally in an intergenic or intronic region of the cell.
   41. The editing system of any one of embodiments 23 to 39, wherein the integration locus is disposed within a coding region of the cell.
   42. The editing system of any one of embodiments 23 to 39, wherein the integration locus is disposed within a noncoding region of the cell.
   43. The editing system of any one of embodiments 23 to 42, wherein the CF editing cassette further comprises an edit to immunize the target locus and prevent re-nicking.
   44. A vector comprising
a nucleic acid sequence encoding a nucleic acid-guided nuclease/reverse transcriptase fusion enzyme;
a nucleic acid sequence encoding a first gRNA having a region of complementarity to a first sequence of an integration locus in a cell;
a nucleic acid sequence encoding a second gRNA having a region of complementarity to a second sequence of the integration locus;
a CF editing cassette comprising from 5′ to 3′;
   a nucleic acid sequence encoding a CFgRNA having a region of complementarity to a sequence of a target locus in the cell, and
   a nucleic acid sequence encoding a repair template;
a 5′ homology arm flanking a 5′ end of the CF editing cassette, the 5′ homology arm having homology to a third sequence of the integration locus; and
a 3′ homology arm flanking a 3′ end of the CF editing cassette, the 3′ homology arm having homology to a fourth sequence of the integration locus.
   45. The vector of embodiment 44, CFgRNA comprises from 5′ to 3′ a spacer region and a structural region recognized by the nucleic acid-guided nuclease/reverse transcriptase fusion enzyme.
   46. The vector of embodiment 44 or 45, wherein the repair template comprises an edit and a primer binding site (PBS).
   47. The vector of embodiment 46, wherein the repair template further comprises post-edit homology region.
   48. The vector of embodiment 46 or 47, wherein the repair template further comprises a nick-to-edit region.
   49. The vector of any one of embodiments 44 to 48, wherein the nucleic acid-guided nuclease/reverse transcriptase fusion enzyme comprises a nucleic acid-guided nickase and a reverse transcriptase.
   50. The vector of embodiment 49, wherein the nucleic acid-guided nickase comprises a MAD nickase or a variant thereof.
   51. The vector of embodiment 49, wherein the nucleic acid-guided nickase comprises a Cas nickase or a variant thereof.
   52. The vector of any one of embodiments 44 to 51, wherein the CF editing cassette further comprises a selectable marker.
   53. The vector of embodiment 52, wherein the selectable marker is for selection and enrichment of cells having an integrated CF editing cassette.
   54. The vector of embodiment 52 or 53, wherein the selectable marker is a puromycin resistance gene.
   55. The vector of any one of embodiments 44 to 54, further comprising self-targeting sequences having complementarity to the first gRNA and/or the second gRNA.
   56. The vector of any one of embodiments 44 to 55, wherein the integration locus is a safe harbor locus disposed centrally in an intergenic or intronic region of the cell.
   57. The vector of any one of embodiments 44 to 55, wherein the integration locus is disposed within a coding region of the cell.
   58. The vector of any one of embodiments 44 to 55 wherein the integration locus is disposed within a noncoding region of the cell.
   59. The vector of any one of embodiments 44 to 58, wherein the CF editing cassette further comprises an edit to immunize the target locus and prevent re-nicking.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: GFP to BFP Conversion Assay

A GFP to BFP reporter cell line is created using mammalian cells with a stably integrated genomic copy of the GFP gene (HEK293T-GFP). These cell lines enable phenotypic detection of genomic edits of different classes by various different mechanisms, including flow cytometry, fluorescent cell imaging, and genotypic detection by sequencing of the genome-integrated GFP gene. Lack of editing, or perfect repair of cut events in the GFP gene, result in cells that remain GFP-positive. Cut events that are repaired by the Non-Homologous End-Joining (NHEJ) pathway often result in nucleotide insertion or deletion events (indels), resulting in frame-shift mutations in the coding sequence that cause loss of GFP gene expression and fluorescence. Cut events that are repaired by the Homology-Directed Repair (HDR) pathway using the GFP to BFP HDR donor as a repair template or by the use of CFgRNAs, e.g., complementary CFgRNAs, result in conversion of the cell fluorescence profile from that of GFP to that of BFP.

### Example II: CREATE Fusion Editing—Proof of Concept MAD2007 Nickase

CREATE Fusion Editing (CFE) is a technique that uses a nucleic acid nickase fusion protein (e.g., MAD2007 nickase) fused to a peptide with reverse transcriptase activity along with a nucleic acid encoding a gRNA comprising a region complementary to a target region of a nucleic acid in one or more cells, which comprises a mutation of at least one nucleotide relative to the target region in the one or more cells and a protospacer adjacent motif (PAM) mutation.

In a first design, a nickase enzyme derived from the MAD2007 nuclease (see, e.g., . U.S. Pat. Nos. 9,982,279 and 10,337,028), e.g., Cas9 H840A nickase or MAD7® nickase (see, e.g., U.S. Ser. Nos. 16/837,212 and 17/084,522), is fused to an engineered reverse transcriptase (RT) on the C-terminus and cloned downstream of a CMV promoter. In this instance, the RT used is derived from Moloney Murine Leukemia Virus (M-MLV).

RNA guides (gRNAs) are designed that are complementary to a single region proximal to the EGFP-to-BFP editing site. The gRNA is extended on the 3′ end to include a region of 13 bp that include the TY-to-SH edit and a second region of 13 bp that is complementary to the nicked EGFP DNA sequence. This allows the nicked genomic DNA to anneal to the 3′ end of the gRNA which can then be extended by the reverse transcriptase to incorporate the edit in the genome. A second gRNA targets a region in the EGFP DNA sequence that is 86 bp upstream of the edit site. This gRNA is designed such that it enables the nickase to cut the opposite strand relative to gRNA. Both of these gRNAs are cloned downstream of a U6 promoter. A poly-T sequence is also included that terminates the transcription of the gRNA.

The plasmids are transformed into NEB Stable E. coli (Ipswich, NY) and grown overnight in 25 mL LB cultures. The following day the plasmids are purified from E. coli using the Qiagen Midi Prep kit (Venlo, Netherlands). The purified plasmid is then RNase A (ThermoFisher, Waltham, Mass) treated and re-purified using the DNA Clean and Concentrator kit (Zymo, Irvine, CA).

HEK293T cells are cultured in DMEM medium which is supplemented with 10% FBS and 1X Penicillin and Streptomycin. 100 ng of total DNA (50 ng of gRNA plasmid and 50 ng of CFE plasmids) is mixed with I μL of PolyFect (Qiagen, Venlo, Netherlands) in 25 μL of OptiMEM in a 96 well plate. The complex is incubated for 10 minutes and then 20,000 HEK293T cells resuspended in 100 μL of DMEM are added to the mixture. The resulting mixture is then incubated for 80 hours at 37° C. and 5% CO2.

The cells are harvested from flat bottom 96 well plates using TrypLE Express reagent (ThermoFisher, Waltham. Mass) and transferred to v-bottom 96 well plate. The plate is then spun down at 500× g for 5 minutes. The TrypLE solution is then aspirated and the cell pellet is resuspended in FACS buffer (1X PBS, 1% FBS, 1mM EDTA and 0.5% BSA). The GFP+, BFP+and RFP+cells are then analyzed on the Attune NxT flow cytometer and the data is analyzed on FlowJo software.

The RFP+BFP+cells that are identified are indicative of the proportion of enriched cells that have undergone precise or imprecise editing process. BFP+cells indicate cells that have undergone successful editing process and express BFP. The GFP-cells indicate cells that have been imprecisely edited, leading to disruption of the GFP open reading frame and loss of expression.

In this experiment, the edit is immediately 3′ of the gRNA, and 3′ of the edit is a further region complementary to the nicked genome, although the intended edit could also be present further 5′ within the region homologous to the nicked genome. A nickase RT fusion enzyme (Cas9 H840A nickase) creates a nick in the target site and the nicked DNA anneals to its complementary sequence on the 3′ end of the gRNA. The RT then extends the DNA, thereby incorporating the intended edit directly in the genome.

The effectiveness of CREATE Fusion Editing in GFP+HEK293T cells is tested. In the assay system devised, a successful precise edit results in a BFP+cell whereas an imprecisely edit turns the cell both BFP and GFP negative. CREATE Fusion gRNA in combination with CFE2.1 or CFE2.2 gives ˜40-45% BFP+cells indicating that almost half the cell population undergoes successful editing (data not shown). The GFP-cells are ˜10% of the population. The use of a second nicking gRNA, as described in Anzalone et al. (Nature, 576 (7785):149-157 (2019)) does not increase the precision edit rate any further; in fact, it significantly increases the imprecisely edited, GFP-negative cell population and the editing rate is lower.

Previous literature has shown that double nicks on opposite strands (<90 bp away) do result in a double strand break which tend to be repaired via NHEJ resulting in imprecise insertions or deletions. Overall, the results indicate that CREATE Fusion Editing predominantly yields precisely edited cells and that the imprecisely edited cells proportion is much lower (data not shown).

An enrichment handle, specifically a fluorescent reporter (RFP) linked to nuclease expression is included in this experimentation as a proxy for cells receiving the editing machinery. When only the RFP-positive cells are analyzed (computational enrichment) after 3 to 4 cell divisions, up to 75% of the cells are BFP+when tested with gRNA (data not shown), indicating uptake or expression-linked reporters can be used to enrich for a population of cells with higher rates of CREATE Fusion-mediated gene editing. In fact, the combined use of CREATE Fusion Editing and the described enrichment methods result in a significantly improved rate of intended edits (data not shown).

### Example III: CREATE Fusion Editing—Proof of Concept

CREATE Fusion Editing is carried out in mammalian cells using a single guide RNA covalently linked to a homology arm having an intended edit to the native sequence and an edit that disrupts nuclease cleavage at this site. Briefly, lentiviral vectors are produced using the following protocol: 1000 ng of lentiviral transfer plasmid containing the CREATE Fusion cassettes along with 1500 ng of lentiviral packaging plasmids (ViraSafe Lentivirus Packaging System Cell BioLabs) are transfected into HEK293T cells using Lipofectamine LTX in 6-well plates. Media containing the lentivirus is collected 72 hours post transfection. Two clones of a lentiviral CREATE Fusion gRNA-HA design are chosen, and an empty lentiviral backbone is included as negative control.

The day before the transduction, 200,000 HEK293T cells are seeded in six well plates. Different volumes of CREATE lentivirus (10 μL to 1000 μL) are added to HEK293T cells in six well plates along with 10 μg/mL of Polybrene. 48 hours after transduction, media with 15 μg/mL of Blasticidin is added to the wells. Cells are maintained in selection for one week. Following selection, the well with lowest number of surviving cells is selected for future experiments (<5% cells).

The experimental constructs or wild-type SpCas9 are electroporated into HEK293T cells using the Neon Transfection System (Thermo Fisher Scientific, Waltham, MA). Briefly, 400 ng of total plasmid DNA is mixed with 100,000 cells in Buffer R in a total of 15 μL volume. The 10 μL Neon tip is used to electroporate cells using 2 pulses of 20 ms and 1150 v. Cells are analyzed on the flow cytometer 80 hours post electroporation. Unenriched editing rates of up to 15% are achieved from single copy delivery of gRNA (data not shown).

When the editing is combined with computational selection of RFP+cells, however, enriched editing rates of up to 30% are achieved from a single copy delivery gRNA. This enrichment via selection of cells receiving the editing machinery is shown to result in a 2-fold increase in precise, complete intended edits (data not shown). Two or more enrichment/delivery steps can also be used to achieve higher editing rates of CREATE Fusion Editing in an automated instrument, e.g., use of a module for cell handle enrichment and identification of cells having BFP expression. When the method enriches for cells that have higher gRNA expression levels, the editing rate is even further increased, and thus a growth and/or enrichment module of the instrument may include gRNA enrichment.

### Example IV: CREATE Fusion Editing—Integration of the GFP-to-BFP Editing Cassette in Induced Pluripotent Stem Cells (iPSCs)

iPSC-GFP cells comprising a stably integrated genomic copy of the GFP gene are transfected with an editing vector as described in FIG. 1D, where the CFgRNA and repair template target a GFP-to-BFP edit, the selectable marker is a puromycin resistance gene, and gRNAI, gRNA2, and the homology arms target various genomic sites or loci (x-axis) for integration of the CF editing cassette (encoding the CFgRNA, replair template, and selectable marker) (FIG. 1F). Successful HDR-driven integration of the CF editing cassette results in the stable integration of the puromycin resistance marker. Before selection with puromycin, the GFP-to-BFP edit rate is <1%. After selection with puromycin for integration of the CF editing cassette, the GFP-to-BFP editing rates are dramatically enriched. The y-axis shows the quantification of BFP+frequency.

While this invention is satisfied by aspects in many different forms, as described in detail in connection with preferred aspects of the invention, it is understood that the present disclosure is to be considered as an example of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

1. A method for performing nucleic acid-guided nuclease/reverse transcriptase fusion editing in a genome of a live cell, comprising:
(a) providing the live cell, wherein the live cell comprises a target locus and an integration locus;
(b) providing a nucleic acid-guided nuclease/reverse transcriptase fusion enzyme;
(c) providing a first guide RNA (gRNA) having a region of complementarity to a first sequence of the integration locus;
(d) providing a second gRNA having a region of complementarity to a second sequence of the integration locus;
(e) providing an editing vector, the editing vector comprising:
(i) a CF editing cassette comprising from 5′ to 3′:
(A) a nucleic acid sequence encoding a CFgRNA having a region of complementarity to a sequence of the target locus, and
(B) a nucleic acid sequence encoding a repair template [5′3′];
(ii) a 5′ homology arm flanking a 5′ end of the CF editing cassette, the 5′ homology arm having homology to a third sequence of the integration locus; and
(iii) a 3′ homology arm flanking a 3′ end of the CF editing cassette, the 3′ homology arm having homology to a fourth sequence of the integration locus;
(f) providing conditions to allow the nucleic acid-guided nuclease/reverse transcriptase fusion enzyme, the CFgRNA, and the repair template to bind to the target locus;
(g) allowing the nucleic acid-guided nuclease/reverse transcriptase fusion enzyme, the CFgRNA, and the repair template to edit the target locus;
(h) providing conditions to allow the nucleic acid-guided nuclease/reverse transcriptase fusion enzyme and the first and second gRNAs to bind and nick at the integration locus; and
(i) allowing the CF editing cassette to integrate into the integration locus.

2. The method of claim 1, further comprising:
sequencing the genome or a transcriptome of the cell to track for integration of the CF editing cassette, the integration of the CF editing cassette representing a nucleic acid-guided nickase/reverse transcriptase fusion editing event.

3. The method of claim 1, further comprising providing an engine vector comprising a nucleic acid sequence encoding the nucleic acid-guided nuclease/reverse transcriptase fusion enzyme, wherein the engine vector is different from the editing vector.

4. The method of claim 1, wherein the CF editing cassette further comprises a selectable marker.

5. The method of claim 4, further comprising selecting and enriching for cells having an integrated CF editing cassette.

6. The method of claim 1, wherein the editing vector further comprises self-targeting sequences having complementarity to the first gRNA and/or the second gRNA.

7. The method of claim 1, wherein the CF editing cassette further comprises an edit to immunize the target locus and prevent re-nicking.

8. An editing system comprising one or more vectors comprising:
a nucleic acid sequence encoding a nucleic acid-guided nuclease/reverse transcriptase fusion enzyme;
a nucleic acid sequence encoding a first gRNA having a region of complementarity to a first sequence of an integration locus in a cell;
a nucleic acid sequence encoding a second gRNA having a region of complementarity to a second sequence of the integration locus;
a CF editing cassette comprising from 5′ to 3′:
a nucleic acid sequence encoding a CFgRNA having a region of complementarity to a sequence of a target locus in the cell, and
a nucleic acid sequence encoding a repair template;
a 5′ homology arm flanking a 5′ end of the CF editing cassette, the 5′ homology arm having homology to a third sequence of the integration locus; and
a 3′ homology arm flanking a 3′ end of the CF editing cassette, the 3′ homology arm having homology to a fourth sequence of the integration locus.

9. The editing system of claim 8, wherein the one or more vectors comprise an editing vector, and wherein the editing vector comprises the CF editing cassette, the 5′ homology arm, and the 3′ homology arm.

10. The editing system of claim 9, wherein the editing vector further comprises a nucleic acid sequence encoding the nucleic acid-guided nuclease/reverse transcriptase fusion enzyme.

11. The editing system of claim 9, wherein the editing vector further comprises a nucleic acid sequence encoding the first gRNA and a nucleic acid sequence encoding the second gRNA.

12. The editing system of claim 9, wherein the editing vector further comprises self-targeting sequences having complementarity to the first gRNA and/or the second gRNA.

13. The editing system of claim 8, wherein the one or more vectors comprise an engine vector, and wherein the engine vector comprises a nucleic acid sequence encoding the nucleic acid-guided nuclease/reverse transcriptase fusion enzyme.

14. The editing system of claim 13, wherein the engine vector further comprises a nucleic acid sequence encoding the first gRNA and a nucleic acid sequence encoding the second gRNA.

15. The editing system of claim 8, wherein the CF editing cassette further comprises a selectable marker.

16. The editing system of claim 8, wherein the CF editing cassette further comprises an edit to immunize the target locus and prevent re-nicking.

17. A vector comprising a nucleic acid sequence encoding a nucleic acid-guided nuclease/reverse transcriptase fusion enzyme;
a nucleic acid sequence encoding a first gRNA having a region of complementarity to a first sequence of an integration locus in a cell;
a nucleic acid sequence encoding a second gRNA having a region of complementarity to a second sequence of the integration locus;
a CF editing cassette comprising from 5′ to 3′:
a nucleic acid sequence encoding a CFgRNA having a region of complementarity to a sequence of a target locus in the cell, and
a nucleic acid sequence encoding a repair template;
a 5′ homology arm flanking a 5′ end of the CF editing cassette, the 5′ homology arm having homology to a third sequence of the integration locus; and
a 3′ homology arm flanking a 3′ end of the CF editing cassette, the 3′ homology arm having homology to a fourth sequence of the integration locus.

18. The vector of claim 17, wherein the CF editing cassette further comprises a selectable marker.

19. The vector of claim 17, further comprising self-targeting sequences having complementarity to the first gRNA and/or the second gRNA.

20. The vector of claim 17, wherein the CF editing cassette further comprises an edit to immunize the target locus and prevent re-nicking.
